(19) United States
(12) Patent Application Publication
(10) Pub. No.: US 2008/0014633 A1
(43) Pub. Date: Jan. 17, 2008

(54) Manipulation of plant senescence using modified promoters

(75) Inventors: German Spangenberg, Bundoora (AU); Carl McDonald Ramage, Craigieburn (AU); Melissa Ann Palviainen, Eden Park (AU); Roger W. Parish, Warrandyte (AU); Joshua Heazlewood, Duncraig (AU)

Correspondence Address: SHELDON MAK ROSE & ANDERSON PC, 100 East Corson Street, Third Floor, PASADENA, CA, 91103-3842, US

(73) Assignees: Agriculture Victoria Services Pty Ltd., Attwood (AU); La Trobe University, Bundoora (AU)

(21) Appl. No.: 11/789,526

(22) Filed: Apr. 24, 2007

Related U.S. Application Data
(63) Continuation-in-part of application No. 10/363,723, filed on Jun. 6, 2003, now Pat. No. 7,227,055.

(30) Foreign Application Priority Data
Sep. 6, 2000 (AU) PQ 9946

(51) Int. Cl. A01H 5/00; C12N 15/00

(52) U.S. Cl. 435/320.1; 800/278; 800/298

(57) ABSTRACT

The present invention relates to methods of manipulating senescence in plants. The invention also relates to vectors useful in such methods, transformed plants with modified senescence characteristics and plant cells, seeds and other parts of such plants.

This application is a continuation-in-part of U.S. patent application Ser. No. 10/363,723, entitled “Manipulation of plant senescence using an MYB gene promoter and cytokinin biosynthesis gene” filed Mar. 5, 2003, which claims priority from International Patent Application PCT/AU01/01092, entitled “Manipulation of Plant Senescence Using An MYB Gene Promoter and Cytokinin Biosynthesis Genes,” filed Aug. 30, 2001, which claims priority from Australian Patent Application PQ 9946, entitled “Manipulation of Plant Senescence Using An MYB Gene Promoter and Cytokinin Biosynthesis Genes,” filed Sep. 6, 2000; the contents of which are incorporated by reference herein in their entirety.

The present invention relates to methods of manipulating senescence in plants. The invention also relates to vectors useful in such methods, transformed plants with modified senescence characteristics and plant cells, seeds and other parts of such plants.

Leaf senescence involves metabolic and structural changes in cells prior to cell death. It also involves the recycling of nutrients to actively growing regions.

The regulation of plant and plant organ senescence by cytokinins has important agricultural consequences. Elevated cytokinin levels in leaves tend to retard senescence. A number of promoters have been used to regulate the expression of the ipt gene, whose product (isopentenyltransferase) catalyses a key step in cytokinin synthesis. However, in general, transgenic plants over-expressing the ipt gene have been reported to have retarded root and shoot growth, no root formation, reduced apical dominance, and reduced leaf area.

It is an object of the present invention to overcome, or at least alleviate, one or more of the difficulties or deficiencies associated with the prior art.

In one aspect, the present invention provides a method of manipulating senescence in a plant, said method including introducing into said plant a genetic construct including a modified myb gene promoter, or a functionally active fragment or variant thereof, operatively linked to a gene encoding an enzyme involved in biosynthesis of a cytokinin, or a functionally active fragment or variant thereof.

The manipulation of senescence relates to the plant and/or specific plant organs. Senescence of different plant organs, such as leaves, roots, shoots, stems, tubers, flowers, stolons, and fruits may be manipulated. The manipulation of plant and plant organ senescence may have important agricultural consequences, such as increased shelf life of e.g. fruits, flowers, leaves and tubers in horticultural produce and cut flowers, reduced perishability of horticultural crops, increased carbon fixation in senescence-retarded leaves leading to enhanced yields, enhanced biomass production in forage plants, enhanced seed production, etc.

“Manipulating senescence” generally relates to delaying senescence in the transformed plant relative to an untransformed control plant. However, for some applications it may be desirable to promote or otherwise modify senescence in the plant. Senescence may be promoted or otherwise modified for example, by utilizing an antisense gene.

An effective amount of said genetic construct may be introduced into said plant, by any suitable technique, for example by transduction, transfection or transformation. By “an effective amount” is meant an amount sufficient to result in an identifiable phenotypic trait in said plant, or a plant, plant seed or other plant part derived therefrom. Such amounts can be readily determined by an appropriately skilled person, taking into account the type of plant, the route of administration and other relevant factors. Such a person will readily be able to determine a suitable amount and method of administration. See, for example, Maniatis et al, Molecular Cloning: A Laboratory Manual, Cold Spring Harbor Laboratory, Cold Spring Harbor, the entire disclosure of which is incorporated herein by reference.

By a “modified myb gene promoter” is meant a promoter normally associated with a myb gene, which promoter is modified to delete or inactivate one or more root specific motifs and/or pollen specific motifs in said promoter.

While applicant does not wish to be restricted by theory, it is postulated that deletion or inactivation of one or more root specific motifs in said myb gene promoter may alleviate or overcome the problem of leaky expression of the gene encoding a cytokinin biosynthetic enzyme in plant meristems, which may affect root development in some species of plants. It is also postulated that deletion or inactivation of one or more pollen specific motifs in said myb gene promoter may alleviate or overcome the problem of leaky expression of the gene encoding a cytokinin biosynthetic enzyme in pollen, which may affect pollen development in some species of plants.

Preferably the modified myb gene promoter is a modified myb32 gene promoter. Preferably the modified myb gene promoter is from Arabidopsis, more preferably Arabidopsis thaliana.

A suitable promoter which may be modified according to the present invention is described in Li et al., Cloning of three MYB-like genes from Arabidopsis (PGR 99-138) Plant Physiology 121:313 (1999), the entire disclosure of which is incorporated herein by reference.

By a “root specific motif” is meant a sequence of 3-7 nucleotides, preferably 4-6 nucleotides, more preferably 5 nucleotides, which directs expression of an associated gene in the roots of a plant.

Preferably the root specific motif includes a consensus sequence ATATT or AATAT.

Preferably, between one and ten, more preferably between three and eight, even more preferably between five and seven root specific motifs are deleted or inactivated, preferably deleted, in said myb gene promoter.

The root specific motifs may be deleted by removing individual motifs or by removing a fragment of the promoter containing one or more motifs. For example, all or part, of the region between nucleotides 1 and 530, preferably between nucleotides 110 and 530 of the Arabidopsis thaliana myb gene promoter may be deleted.

The deletion may be effected by cutting the nucleic acid, for example with restriction endonucleases, and ligating the cut ends to generate a promoter with a fragment removed.

For example, a modified Arabidopsis thaliana myb gene promoter may be prepared by removing a fragment between the XcmI site at positions 162-176 and the SspI site at positions 520-525. This generates a modified myb gene promoter with 6 of the 7 root specific motifs deleted. Alternatively, all 7 of the root specific motifs may be deleted, for example by deleting the region upstream of the SspI site at positions 520-525, or by deleting the region between nucleotides 1 and 120 together with the region between the XcmI site at positions 162-176 and the SspI site at positions 520-525.

A root specific motif may be inactivated by adding, deleting, substituting or derivatizing one or more nucleotides within the motif, so that it no longer has the preferred consensus sequence.

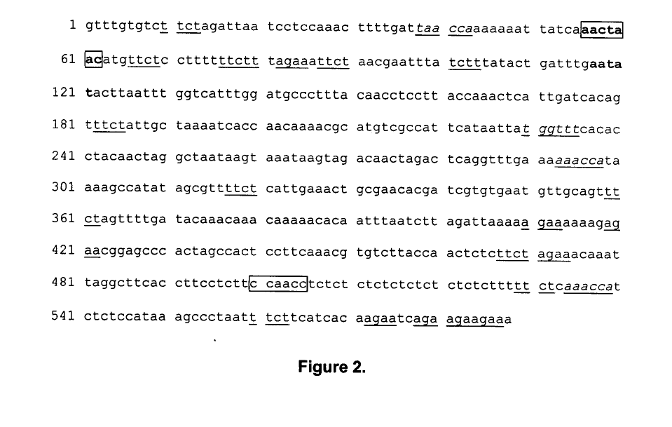

Preferably the modified myb gene promoter includes a nucleotide sequence selected from the group consisting of the sequences shown in FIGS. 2, 3 and 4 hereto (Sequence ID Nos: 2, 3 and 4, respectively) and functionally active fragments and variants thereof.

By a “pollen specific motif” is meant a sequence of 3-7 nucleotides, preferably 4-6 nucleotides, more preferably 4 or 5 nucleotides, which directs expression of an associated gene in the pollen of a plant.

Preferably the pollen specific motif includes a consensus sequence selected from the group consisting of TTCT and AGAA.

Preferably, between one and thirty, more preferably between three and fifteen, even more preferably between four and ten pollen specific motifs are deleted or inactivated, preferably deleted, in said myb gene promoter.

The pollen specific motifs may be deleted by removing individual motifs or by removing a fragment of the promoter containing one or more motifs. For example, all or part of the region between nucleotides 1 and 540, preferably between nucleotides 390 and 540 of the Arabidopsis thaliana myb gene promoter may be deleted.

The deletion may be effected by cutting the nucleic acid, for example with restriction endonucleases, and ligating the cut ends to generate a promoter with a fragment removed.

For example, a modified Arabidopsis thaliana myb gene promoter may be prepared by removing a fragment between the XcmI site at positions 162-176 and the SspI site at positions 520-525. This generates a modified myb gene promoter with 4 of the 23 pollen specific motifs deleted. Alternatively, 10 of the pollen specific motifs may be deleted, for example by deleting the region upstream of the SspI site at positions 520-525.

A pollen specific motif may be inactivated by adding, deleting, substituting or derivatizing one or more nucleotides within the motif, so that it no longer has the preferred consensus sequence.

Preferably the modified myb gene promoter includes a nucleotide sequence selected from the group consisting of the sequences shown in FIGS. 2, 3 and 4 hereto (Sequence ID Nos: 2, 3 and 4, respectively) and functionally active fragments and variants thereof.

In a further aspect of the present invention there is provided a method of enhancing biomass in a plant, said method include introducing into said plant a genetic construct including a myb gene promoter, or a functionally active fragment or variant thereof, operatively linked to a gene encoding an enzyme involved in biosynthesis of a cytokinin, or a functionally active fragment or variant thereof.

The myb gene promoter or a functionally active fragment or variant thereof may be a full length myb gene promoter or a modified myb gene promoter.

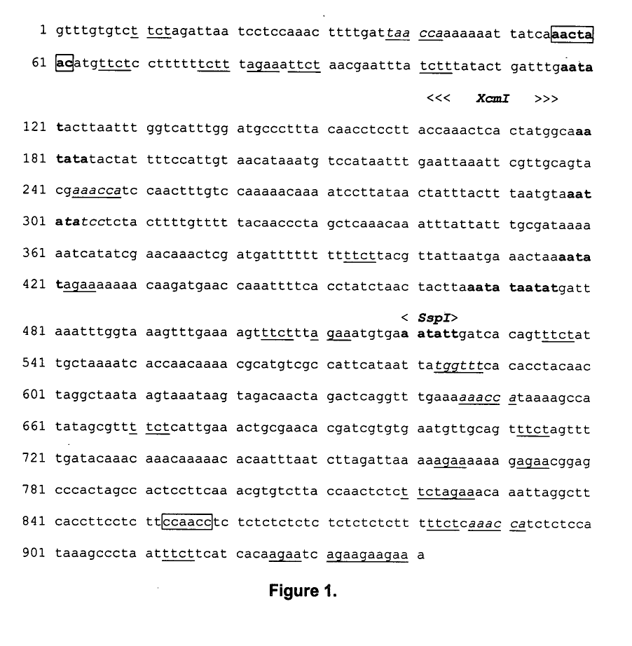

The full length myb gene promoter may be a myb32 gene promoter. Preferably the myb gene promoter is from Arabidopsis, more preferably Arabidopsis thaliana. Most preferably the myb gene promoter includes a nucleotide sequence selected from the group consisting of the sequence shown in FIG. 1 hereto (Sequence ID No: 1) and functionally active fragments and variants thereof.

A suitable promoter is described in Li et al., Cloning of three MYB-like genes from Arabidopsis (PGR 99-138) Plant Physiology 121:313 (1999).

The modified myb gene promoter may be a modified myb gene promoter as hereinbefore described.

By “enhancing biomass” is meant enhancing or increasing in a transformed plant relative to an untransformed control plant a growth characteristic selected from the group consisting of total leaf area, cumulative leaf area, leaf growth dynamics (i.e. number of leaves over time), stolon length, percentage of flowering plants and seed yield per flower or per area sown. “Enhancing biomass” also includes reducing or decreasing percentage stolon death in a transformed plant relative to an untransformed control plant.

In particular, applicants have found that while the seed weight (i.e. weight of thousand seeds) of transgenic plants according to the present invention was indistinguishable from non-transgenic control plants, the total seed yield expressed on the basis of per flower or per area sown was significantly higher in the transgenic plants when compared with non-transgenic control plants of equivalent flowering intensity.

By “functionally active” in relation to a myb gene promoter or modified myb gene promoter is meant that the fragment or variant (such as an analogue, derivative or mutant) is capable of manipulating senescence in a plant by the method of the present invention. Such variants include naturally occurring allelic variants and non-naturally occurring variants. Additions, deletions, substitutions and derivatizations of one or more of the nucleotides are contemplated so long as the modifications do not result in loss of functional activity of the fragment or variant. Preferably the functionally active fragment or variant has at least approximately 80% identity to the relevant part of the above mentioned sequence to which the fragment or variant corresponds, more preferably at least approximately 90% identity, most preferably at least approximately 95% identity. Preferably the fragment has a size of at least 20 nucleotides, more preferably at least 50 nucleotides, more preferably at least 100 nucleotides, more preferably at least 200 nucleotides, most preferably at least 300 nucleotides.

By a “gene encoding an enzyme involved in biosynthesis of a cytokinin” is meant a gene encoding an enzyme involved in the synthesis of cytokines such as kinetin, zeatin and benzyl adenine, for example a gene encoding isopentyl transferase (ipt), or an ipt-like gene such as the sho gene (eg. from petunia). Preferably the gene is an isopentenyl transferase (ipt) gene or sho gene. In a preferred embodiment, the gene is from a species selected from the group consisting of Agrobacterium, more preferably Agrobacterium tumefaciens; Lotus, more preferably Lotus japonicus; and Petunia, more preferably Petunia hybrida.

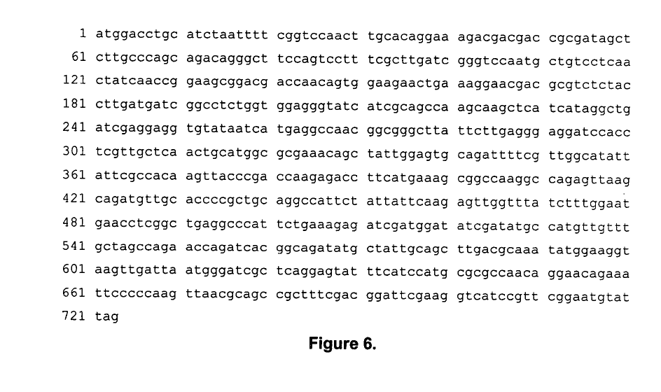

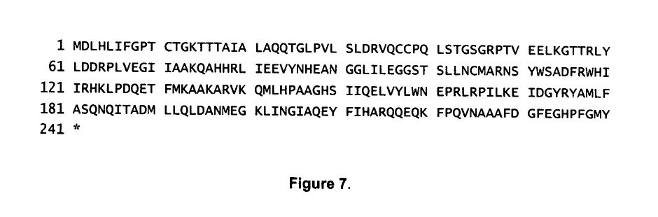

Most preferably the gene includes a nucleotide sequence selected from the group consisting of the sequences shown in FIGS. 6, 8 and 10 hereto (Sequence ID Nos: 5, 7 and 9) sequences encoding the polypeptides shown in FIGS. 7, 9 and 11 hereto (Sequence ID Nos: 6, 8 and 10), and functionally active fragments and variants thereof.

By “functionally active” in relation to a gene encoding a cytokinin biosynthetic enzyme is meant that the fragment or variant (such as an analogue, derivative or mutant) is capable of manipulating senescence in a plant by the method of the present invention. Such variants include naturally occurring allelic variants and non-naturally occurring variants. Additions, deletions, substitutions and derivatizations of one or more of the nucleotides are contemplated so long as the modifications do not result in loss of functional activity of the fragment or variant. Preferably the functionally active fragment or variant has at least approximately 80% identity to the relevant part of the above mentioned sequence, to which the fragment or variant corresponds more preferably at least approximately 90% identity, most preferably at least approximately 95% identity. Such functionally active variants and fragments include, for example, those having conservative nucleic acid changes or nucleic acid changes which result in conservative amino acid substitutions of one or more residues in the corresponding amino acid sequence. For example, the functionally active variant may include one or more conservative nucleic acid substitutions of a sequence shown in FIG. 6, 8 or 10, the resulting functionally active variant encoding an amino acid sequence shown in FIG. 7, 9 or 11, respectively. Preferably the fragment has a size of at least 20 nucleotides, more preferably at least 50 nucleotides, more preferably at least 100 nucleotides, more preferably at least 500 nucleotides.

The genetic construct may be introduced into the plant by any suitable technique. Techniques for incorporating the genetic constructs of the present invention into plant cells (for example by transduction, transfection or transformation) are well known to those skilled in the art. Such techniques include Agrobacterium mediated introduction, electroporation to tissues, cells and protoplasts, protoplast fusion, injection into reproductive organs, injection into immature embryos and high velocity projectile introduction to cells, tissues, calli, immature and mature embryos, biolistic transformation and combinations thereof. The choice of technique will depend largely on the type of plant to be transformed, and may be readily determined by an appropriately skilled person.

Cells incorporating the genetic construct of the present invention may be selected, as described below, and then cultured in an appropriate medium to regenerate transformed plants, using techniques well known in the art. The culture conditions, such as temperature, pH and the like, will be apparent to the person skilled in the art. The resulting plants may be reproduced, either sexually or asexually, using methods well known in the art, to produce successive generations of transformed plants.

The methods of the present invention may be applied to a variety of plants, including monocotyledons [such as grasses (e.g. forage, turf and bioenergy grasses including perennial ryegrass, tall fescue, Italian ryegrass, red fescue, reed canarygrass, big bluestem, cordgrass, napiergrass, wildrye, wild sugarcane, Miscanthus), corn, oat, wheat and barley)], dicotyledons [such as Arabidopsis, tobacco, soybean, clovers (e.g. white clover, red clover, subterranean clover), alfalfa, canola, vegetable brassicas, lettuce, spinach] and gymnosperms.

In a further aspect of the present invention there is provided a vector capable of manipulating senescence in a plant, said vector including a modified myb gene promoter, or a functionally active fragment or variant thereof, operatively linked to a gene encoding an enzyme involved in the biosynthesis of a cytokinin, or a functionally active fragment or variant thereof.

In a still further aspect of the present invention there is provided a vector capable of enhancing biomass in a plant, said vector including a myb gene promoter, or a functionally active fragment or variant thereof, operatively linked to a gene encoding an enzyme involved in the biosynthesis of a cytokinin, or a functionally active fragment or variant thereof.

The myb gene promoter or a functionally active fragment or variant thereof may be a full length myb gene promoter or a modified myb gene promoter, as described herein.

In a preferred embodiment of this aspect of the invention, the vector may further include a terminator; said promoter, gene and terminator being operably linked.

By “operably linked” is meant that said promoter is capable of causing expression of said gene in a plant cell and said terminator is capable of terminating expression of said gene in a plant cell. Preferably, said promoter is upstream of said gene and said terminator is downstream of said gene.

The vector may be of any suitable type and may be viral or non-viral. The vector may be an expression vector. Such vectors include chromosomal, non-chromosomal and synthetic nucleic acid sequences, eg. derivatives of plant viruses; bacterial plasmids; derivatives of the Ti plasmid from Agrobacterium tumefaciens; derivatives of the Ri plasmid from Agrobacterium rhizogenes; phage DNA; yeast artificial chromosomes; bacterial artificial chromosomes; binary bacterial artificial chromosomes; vectors derived from combinations of plasmids and phage DNA. However, any other vector may be used as long as it is replicable or integrative or viable in the plant cell.

The promoter, gene and terminator may be of any suitable type and may be endogenous to the target plant cell or may be exogenous, provided that they are functional in the target plant cell.

A variety of terminators which may be employed in the vectors of the present invention are also well known to those skilled in the art. The terminator may be from the same gene as the promoter sequence or a different gene. Particularly suitable terminators are polyadenylation signals, such as the CaMV 35S polyA and other terminators from the nopaline synthase (nos) and the octopine synthase (ocs) genes.

The vector, in addition to the promoter, the gene and the terminator, may include further elements necessary for expression of the gene, in different combinations, for example vector backbone, origin of replication (ori), multiple cloning sites, spacer sequences, enhancers, introns (such as the maize Ubiquitin Ubi intron), antibiotic resistance genes and other selectable marker genes [such as the neomycin phosphotransferase (nptII) gene, the hygromycin phosphotransferase (hph) gene, the phosphinothricin acetyltransferase (bar or pat) gene], and reporter genes (such as beta-glucuronidase (GUS) gene (gusA)]. The vector may also contain a ribosome binding site for translation initiation. The vector may also include appropriate sequences for amplifying expression.

As an alternative to use of a selectable marker gene to provide a phenotypic trait for selection of transformed host cells, the presence of the vector in transformed cells may be determined by other techniques well known in the art, such as PCR (polymerase chain reaction), Southern blot hybridisation analysis, histochemical assays (e.g. GUS assays), thin layer chromatography (TLC), northern and western blot hybridisation analyses.

Those skilled in the art will appreciate that the various components of the vector are operably linked, so as to result in expression of said gene. Techniques for operably linking the components of the vector of the present invention are well known to those skilled in the art. Such techniques include the use of linkers, such as synthetic linkers, for example including one or more restriction enzyme sites.

In a further aspect of the present invention there is provided a transgenic plant cell, plant, plant seed or other plant part, with modified senescence characteristics or enhanced biomass. Preferably said plant cell, plant, plant seed or other plant part includes a vector according to the present invention. Preferably the transgenic plant cell, plant, plant seed or other plant part is produced by a method according to the present invention.

The present invention also provides a transgenic plant, plant seed or other plant part derived from a plant cell of the present invention.

The present invention also provides a transgenic plant, plant seed or other plant part derived from a plant of the present invention.

The present invention will now be more fully described with reference to the accompanying examples and drawings. It should be understood, however, that the description following is illustrative only and should not be taken in any way as a restriction on the generality of the invention described above.

In the figures:

The patent or application file contains at least one drawing executed in color. Copies of this patent or patent application publication with color drawings will be provide by the Office upon request and payment of the necessary fee.

FIG. 1 shows the nucleotide sequence of the promoter from myb32 gene (atmyb32) from Arabidopsis thaliana (Sequence ID No: 1), Atmyb32 promoter sequence with MYB type, pollen specific and Root specific motifs highlighted. WMCCA (underline/italics) MYB1AT; GTTAGTT (bold/box) MYB1LEPR; CCWACC (box) MYBPZM; GGATA (italics) MYBST1; AGAAA (underline) POLLEN1LELAT52; ATATT (bold) ROOTMOTIFTAPOX1.

FIG. 2 shows an Atmyb32 promoter sequence variant (Atmyb32xs) with the XcmI-SspI plant sequence deleted. MYB type, pollen specific and Root specific motifs are highlighted. WMCCA (underline/italics) MYB1AT; GTTAGTT (bold/box) MYB1LEPR; CCWACC (box) MYBPZM; AGAAA (underline) POLLEN1LELAT52; ATATT (bold) ROOTMOTIFTAPOX1 (Sequence ID No: 2).

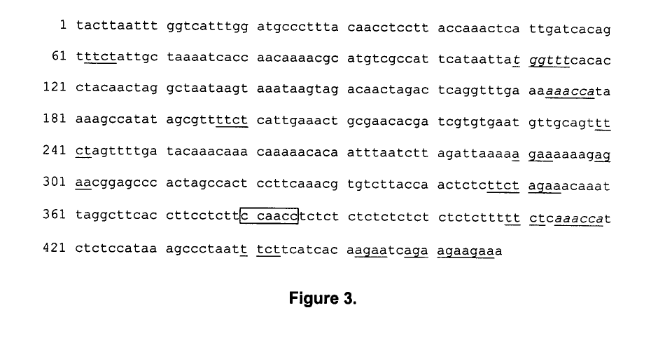

FIG. 3 shows an Atmyb32 promoter variant sequence with all root motifs deleted. MYB type, pollen specific and Root specific motifs are highlighted. WMCCA (underline/italics) MYB1AT; CCWACC (box) MYBPZM; AGAAA (underline) POLLEN1LELAT52 (Sequence ID No: 3)

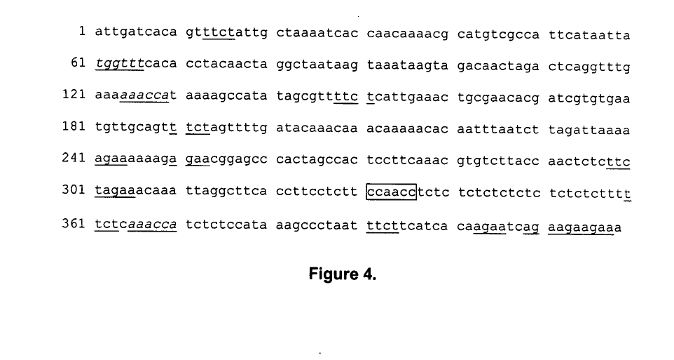

FIG. 4 shows an Atmyb32 promoter variant sequence with the SspI site upstream sequence deleted. MYB type, pollen specific and Root specific motifs are highlighted. WMCCA (underline/italics) MYB1AT; CCWACC (box) MYBPZM; AGAM (underline) POLLEN1LELAT52 (Sequence ID No: 4).

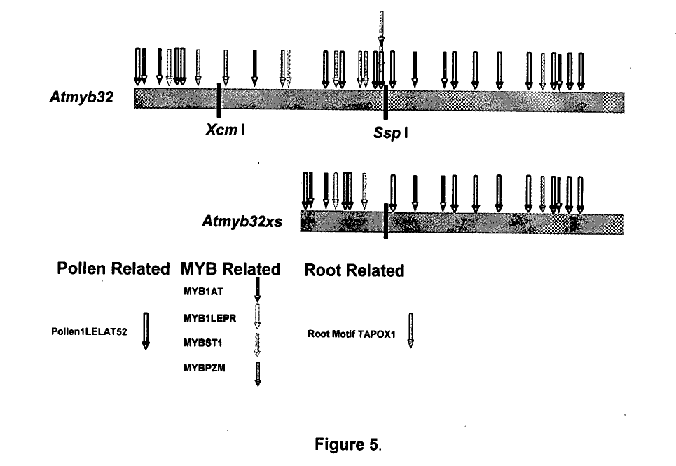

FIG. 5 shows Motifs in Atmyb32 and Atmyb32xs promoter sequences.

FIG. 6 shows the nucleotide sequence of the isopentenyl transferase (ipt) gene from Agrobacterium tumefaciens (Sequence ID No: 5).

FIG. 7 shows the deduced amino acid sequence of the isopentyl transferase gene from Agrobacterium tumefaciens (Sequence ID No. 6)

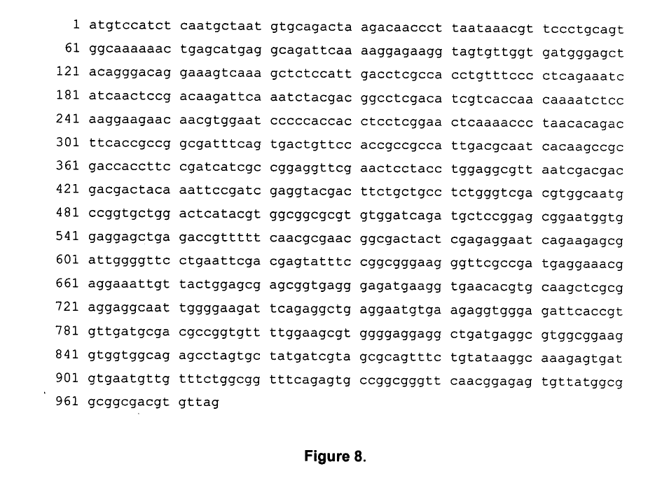

FIG. 8 shows the nucleotide sequence of the isopentyl transferase gene from Lotus japonicus (Sequence ID No. 7).

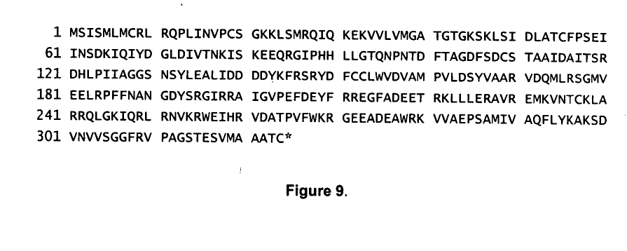

FIG. 9 shows the deduced amino acid sequence of the isopentyl transferase gene from Lotus japonicus (Sequence ID No. 8).

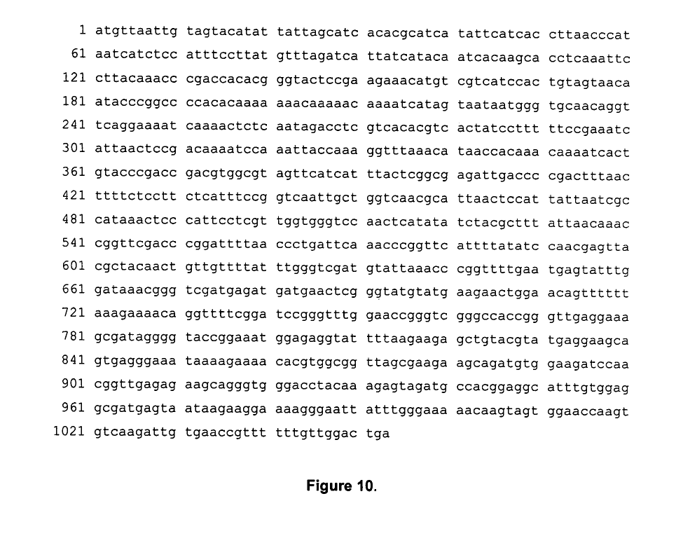

FIG. 10 shows the Nucleotide sequence of the cytokinin biosynthesis Sho gene from Petunia hybrida (Sequence ID No. 9)

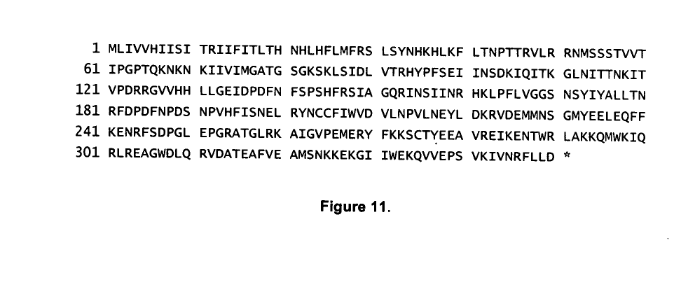

FIG. 11 shows the Deduced amino acid sequence of the cytokinin biosynthesis Sho gene from Petunia hybrida (Sequence ID No. 10).

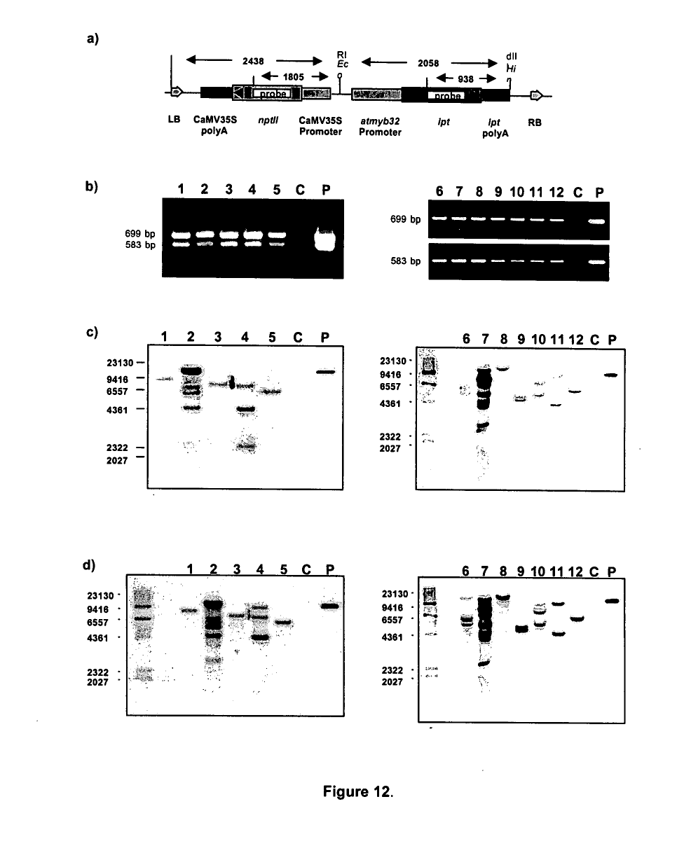

FIG. 12 shows PCR and Southern DNA analysis of atmyb32::ipt transgenic white clover (Trifolium repens) plants. a) The T-DNA region of patmyb32:ipt showing restriction enzyme sites and location of the probes used for Southern hybridization analysis. b) Ethidium bromide stained 1% agarose gel of the PCR amplified 599 bp nptII and 583 bp ipt products. c) Southern blot hybridization with HindIII digested total genomic DNA isolated from PCR positive white clover plants hybridized with the ipt probe. d) Southern blot hybridization with HindIII digested total genomic DNA isolated from PCR positive white clover plants hybridized with the nptII probe. Lanes 1-2: two independent kanamycin resistant cv. Haifa regenerants, code: Hmi01, Hmi08 respectively; Lanes 3-12: twelve independent kanamycin resistant cv. Irrigation regenerants, codes: Imi06, Imi07, Imi08, Imi09, Imi10, Imi11, Imi12, Imi14, Imi16, Imi18 respectively; Lane C: non-transformed white clover; Lane P: positive control plasmid patmyb32ipt.

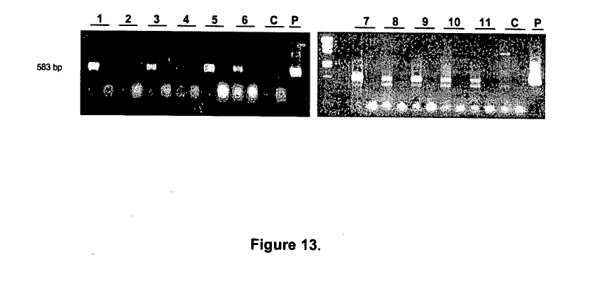

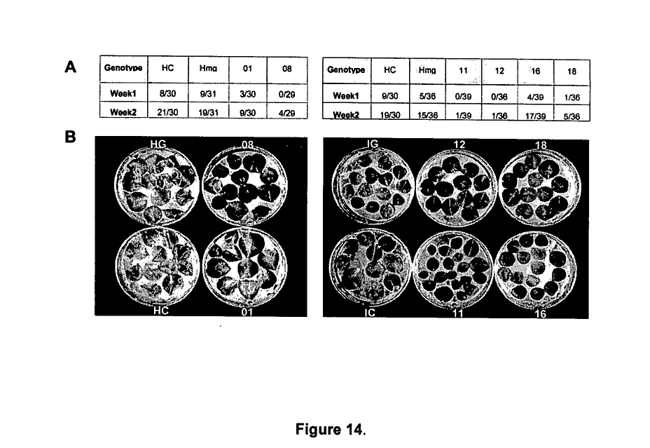

FIG. 13 shows RT-PCR analysis of ipt mRNA expression in atmyb32::ipt transgenic white clover (T. repens) plants. Lane 1-11 are samples from 11 independent transgenic lines with corresponding plant codes as in FIG. 4.8; Lane C, Control non-transformed plant; Lane P, plasmid as positive control. Total RNA was isolated from leaf tissues. Total RNA (13 μg) was used for each reverse transcription reaction and ⅕ of RT product was amplified by PCR. DNA products on the gel on the right were amplified by 2×30 cycles intensive PCR. No reverse transcriptase was added to the corresponding RT-PCR reaction loaded into alternate lanes.

FIG. 14 shows a senescence bioassay of excised leaves from atmyb32::ipt transgenic white clover (T. repens) plants. At least 30 leaves were collected from each line from similar positions on stolons of plant lines. A. The number of yellowing leaves as a fraction of the total number of excised leaves. B. Typical appearance of leaves kept on water under light for two weeks. Key to plant lines: HC, IC and Hmg, 1 mg, non-transformed and atmyb32::gusA transgenic plants (cv. Haifa and Irrigation) respectively; 01 and 08, atmyb32::ipt transgenic Haifa lines Hmi01 and Hmi08 respectively; 11, 12, 16 and 18 atmyb32::ipt transgenic Irrigation lines Imi11, Imi12, Imi16 and Imi18 respectively.

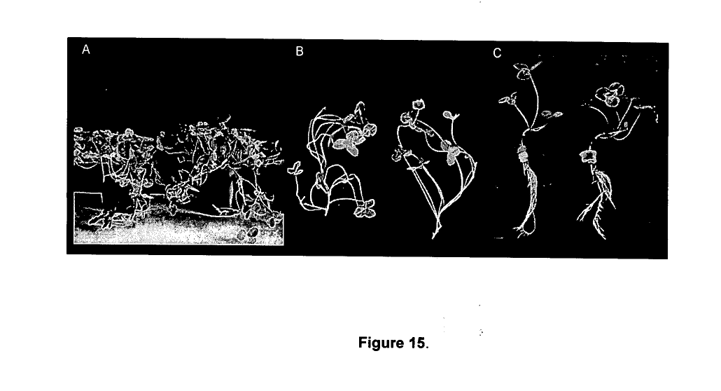

FIG. 15 shows A) General plant morphology, B) Normal shoot development, and C) Normal root development in atmyb32::ipt transgenic white clover (T. repens) (right) plants compared to control plants (left).

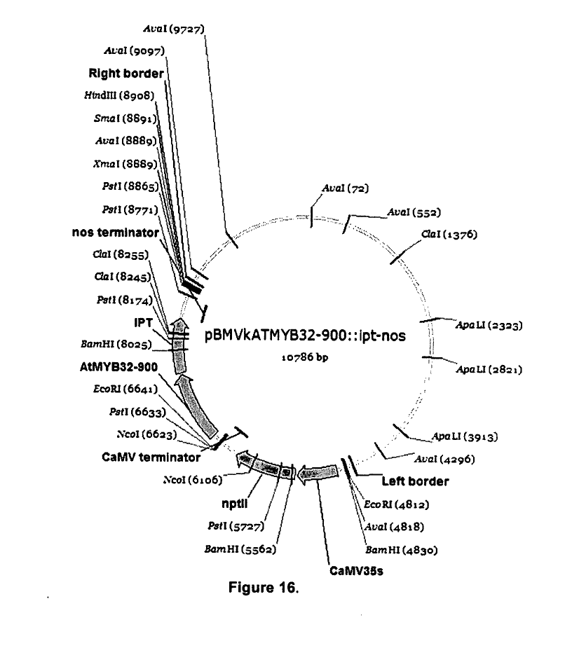

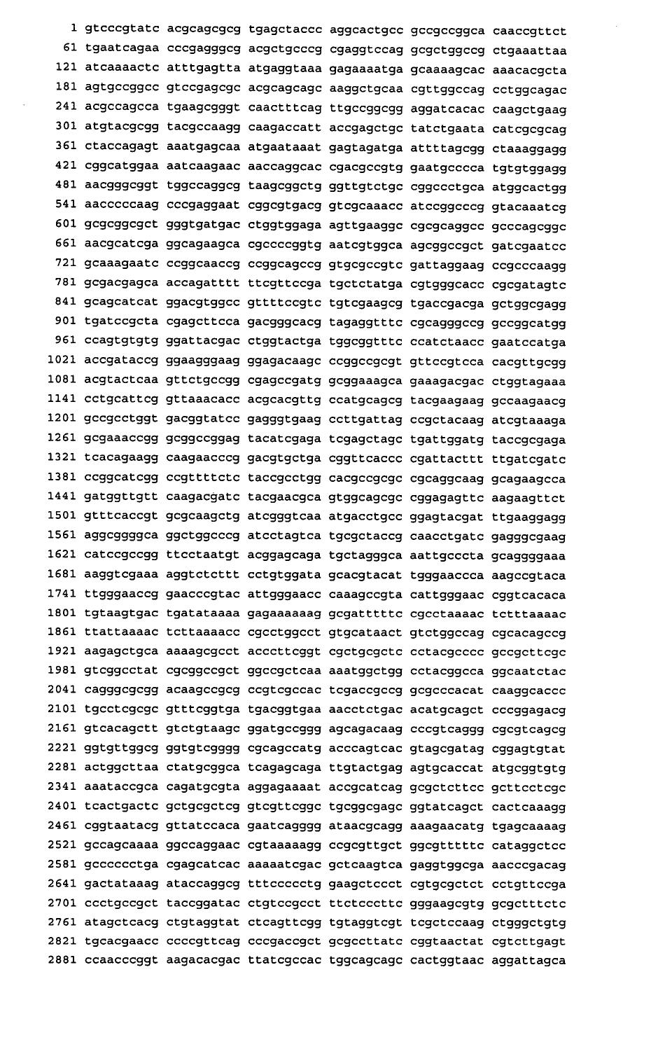

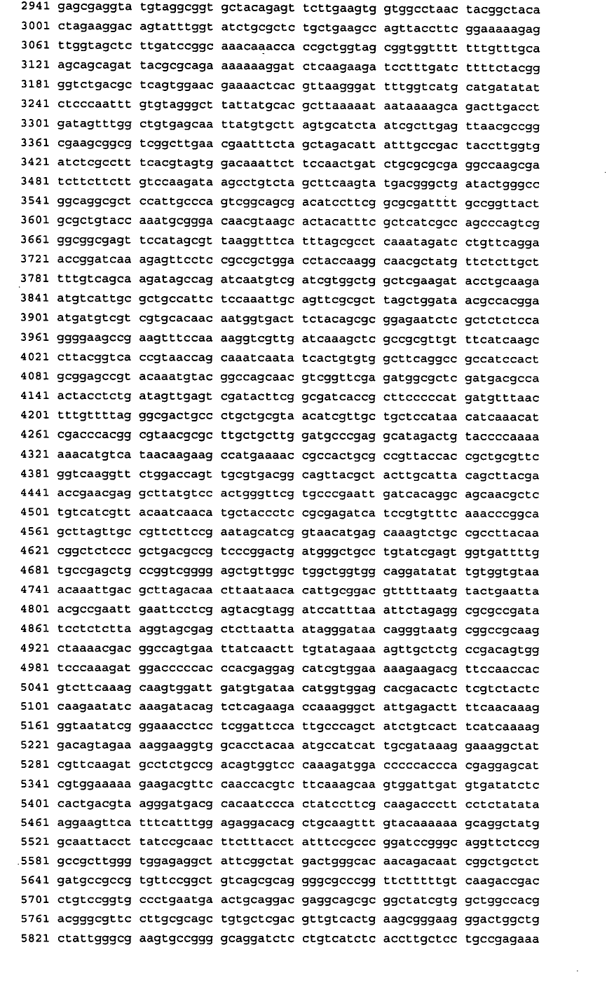

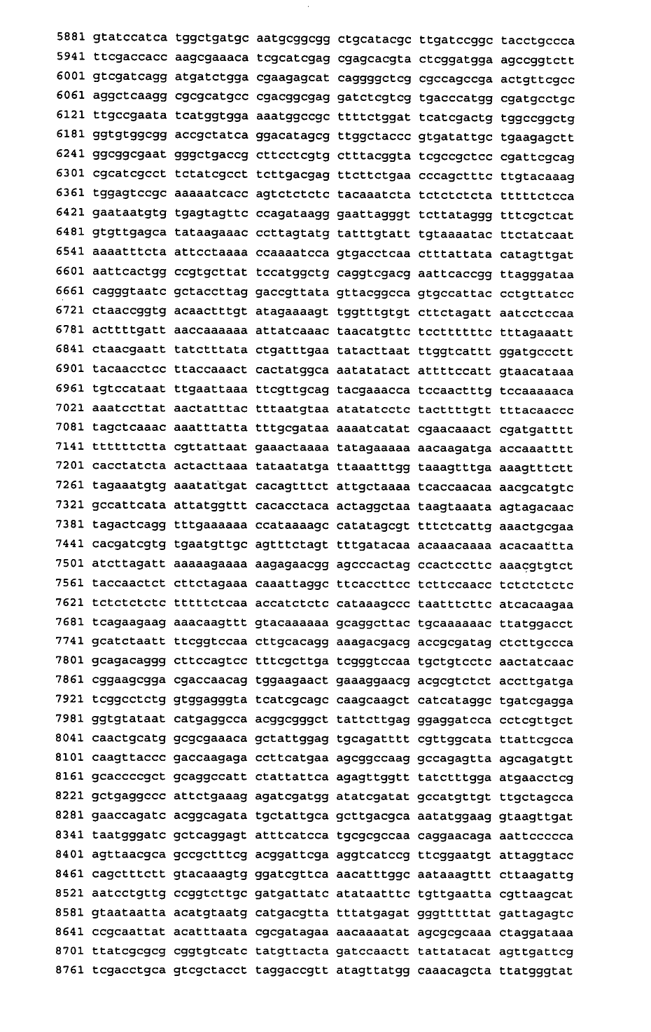

FIG. 16 shows vector details for pBMVkAtMYB32-900::ipt [Gene: Isopentyl transferase (IPT); Vector: pBMVkAtMYB32-900::ipt-nos (backbone pPZPRCS2); Selectable marker: spec; Plant selectable marker cassette: 35S::kan::35ST; Gene promoter: AtMYB32-900; Gene terminator: nos.]

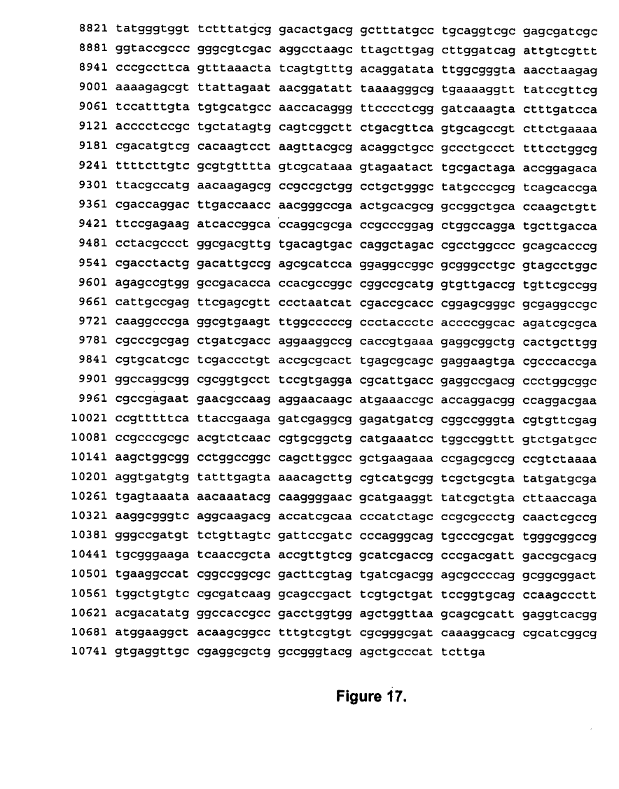

FIG. 17 shows nucleotide sequence of vector pBMVkAtMYB32-900::ipt-nos (Sequence ID No. 11)

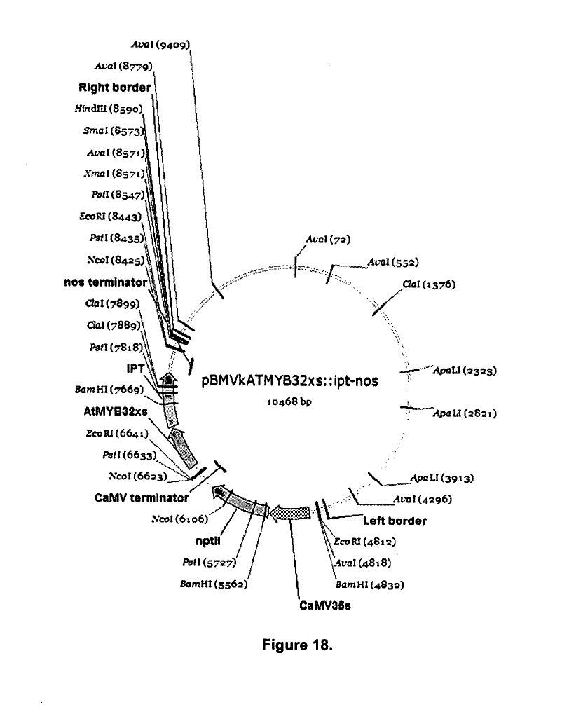

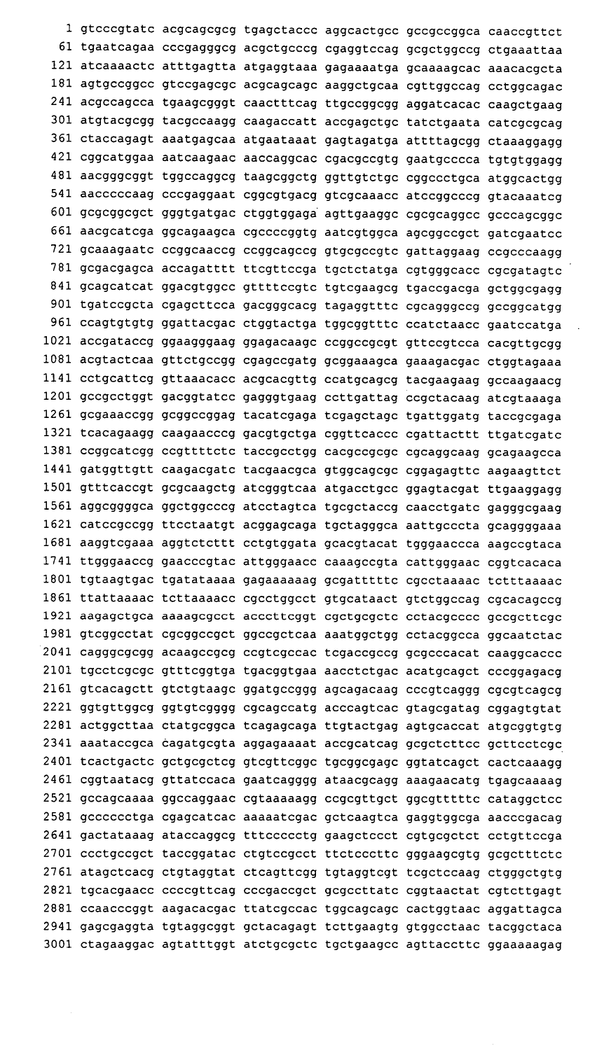

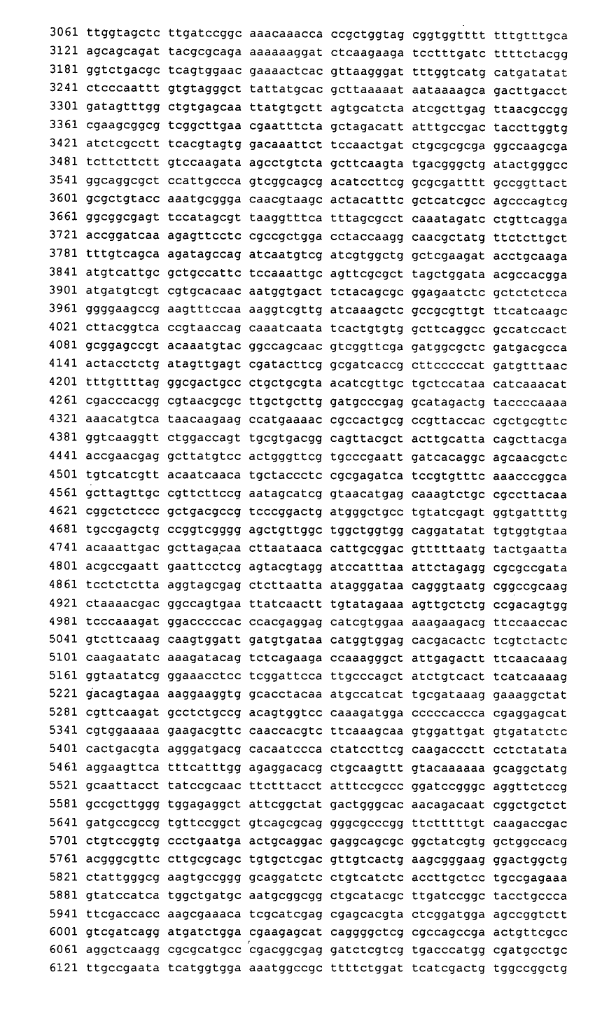

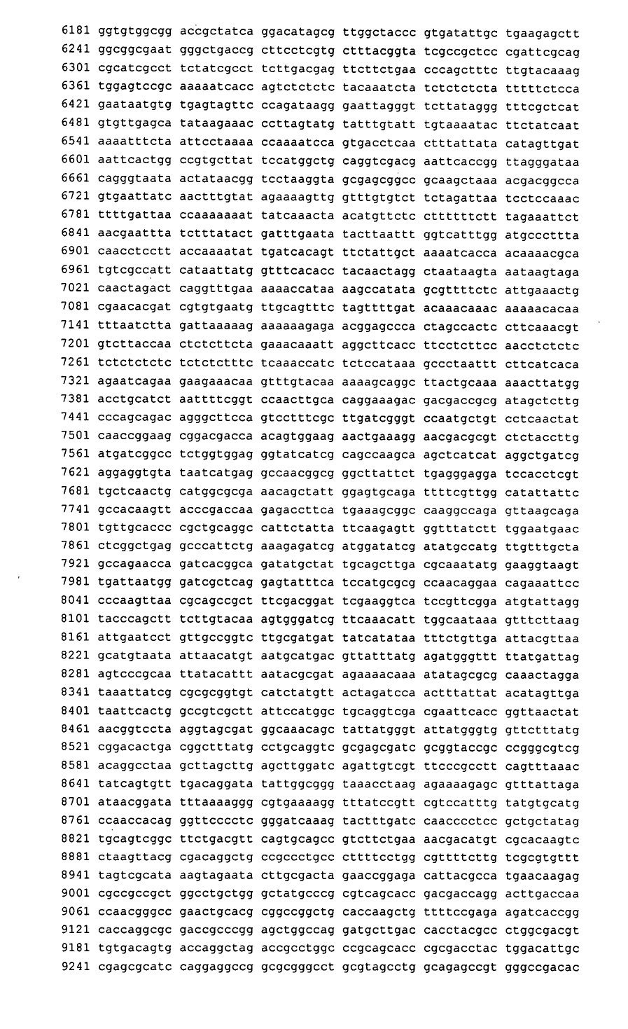

FIG. 18 shows vector details for pBMVkAtMYB32xs::ipt-nos [Gene: Isopentyl transferase (IPT); Vector: pBMVkAtMYB32XS::ipt-nos (backbone pPZPRCS2); Selectable marker: spec; Plant selectable marker cassette: 35S::kan::35ST; Gene promoter: AtMYB32-xs; Gene terminator: nos.]

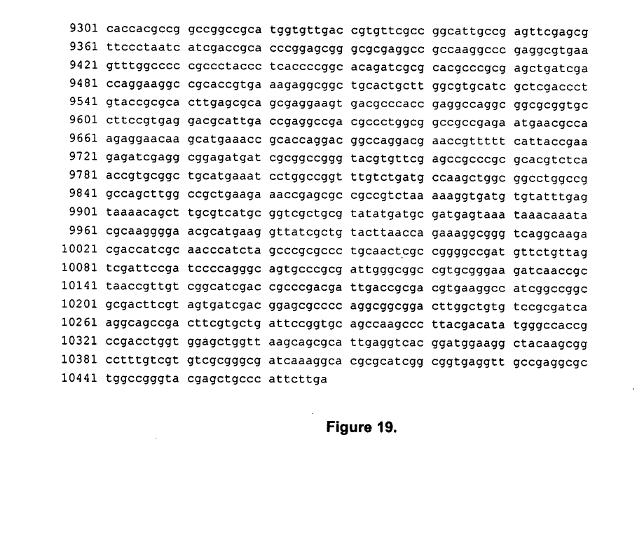

FIG. 19 shows nucleotide sequence of vector pBMVkAtMYB32xs::ipt-nos (Sequence ID No. 12).

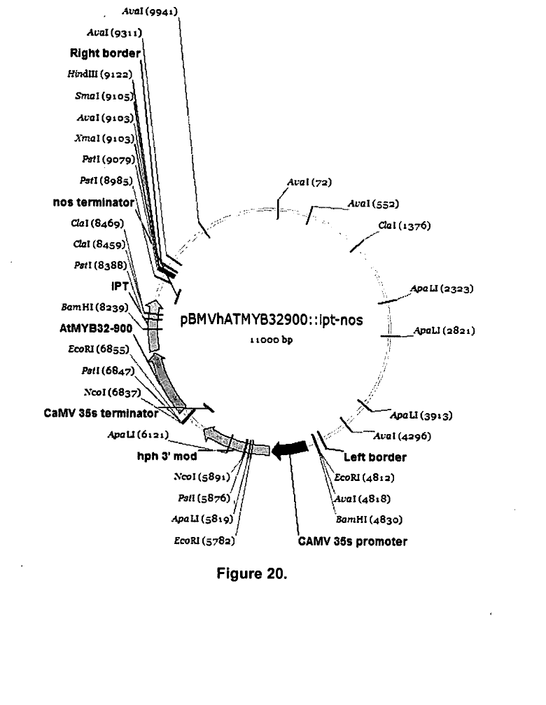

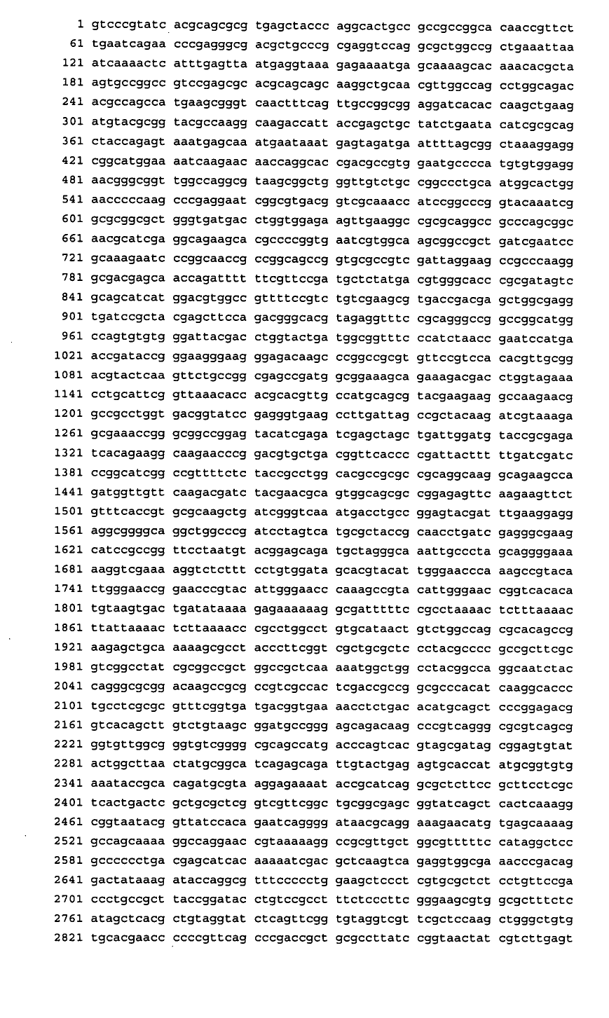

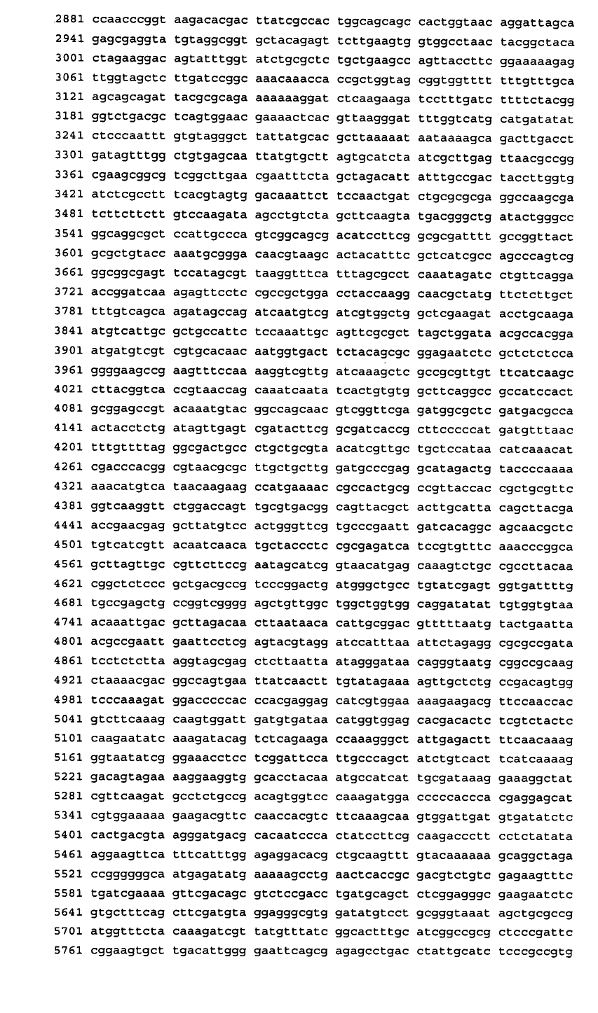

FIG. 20 shows vector details for pBMVhAtMYB32-900::ipt-nos [Gene: Isopentyl transferase (IPT); Vector: pBMVhAtMYB32-900::ipt-nos (backbone pPZPRCS2); Selectable marker: spec; Plant selectable marker cassette: 35S::hph::35ST; Gene promoter: AtMYB32-900; Gene terminator: nos.]

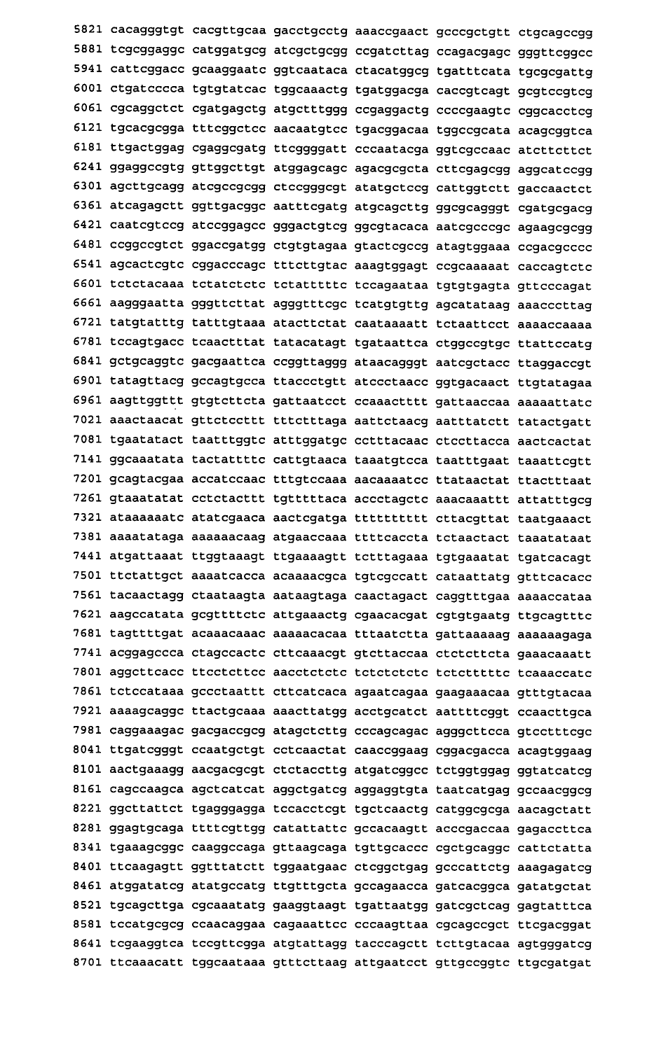

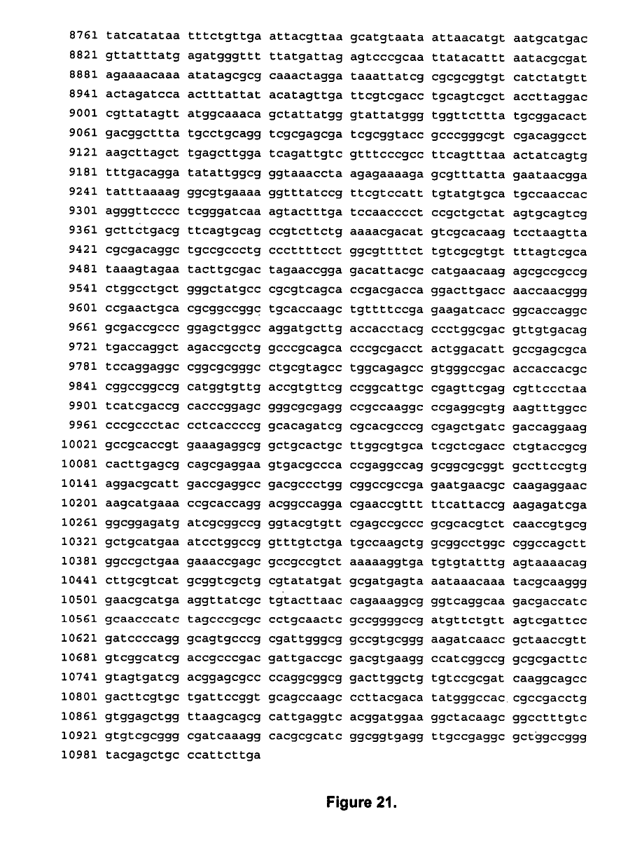

FIG. 21 shows nucleotide sequence of vector pBMVhAtMYB32-900::ipt-nos (Sequence ID No. 13)

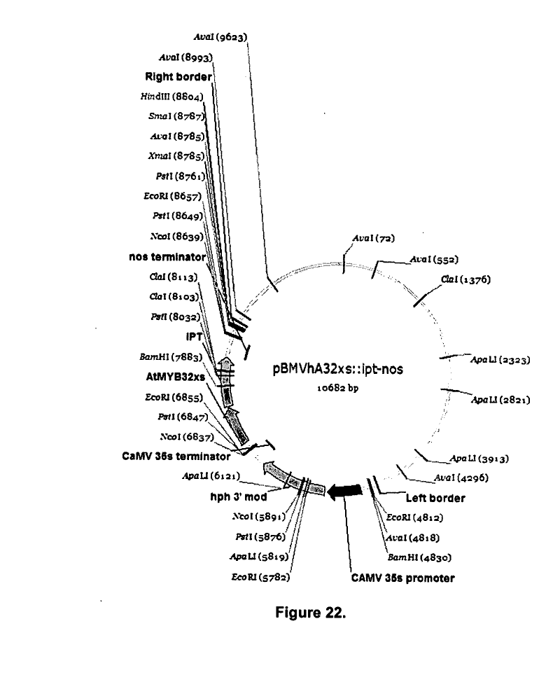

FIG. 22 shows vector details for pBMVhAtMYB32xs::ipt-nos [Gene: Isopentyl transferase (IPT); Vector: pBMVhAtMYB32XS::ipt-nos (backbone pPZPRCS2); Selectable marker: spec; Plant selectable marker cassette: 35S::hph::35ST; Gene promoter: AtMYB32-xs; Gene terminator: nos.]

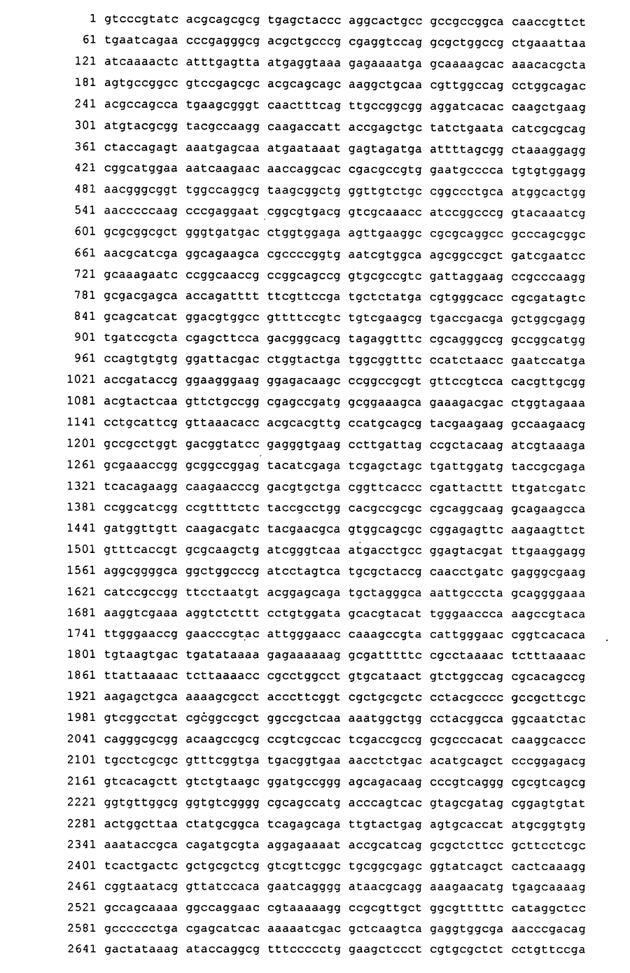

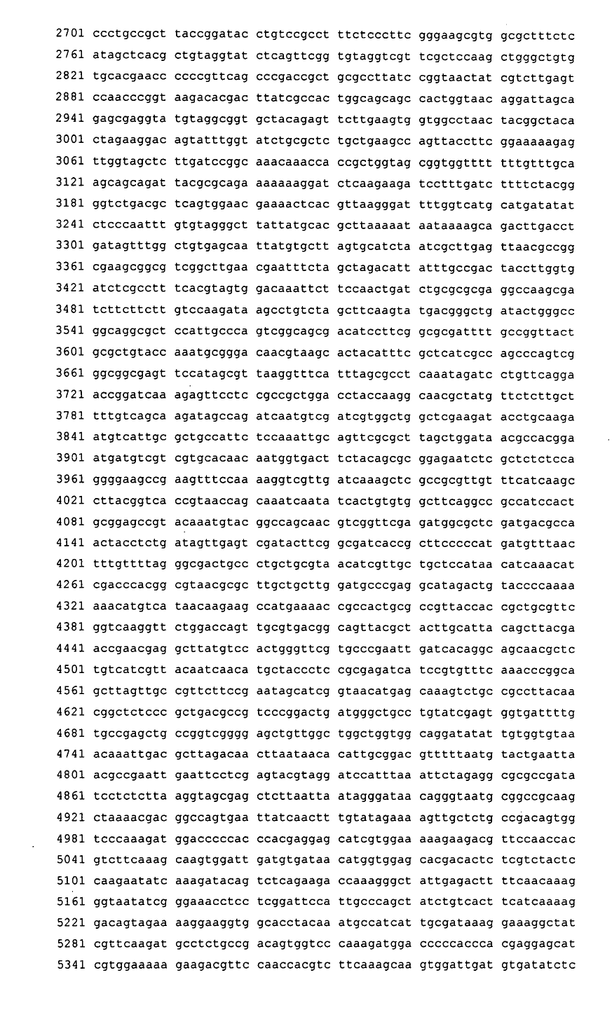

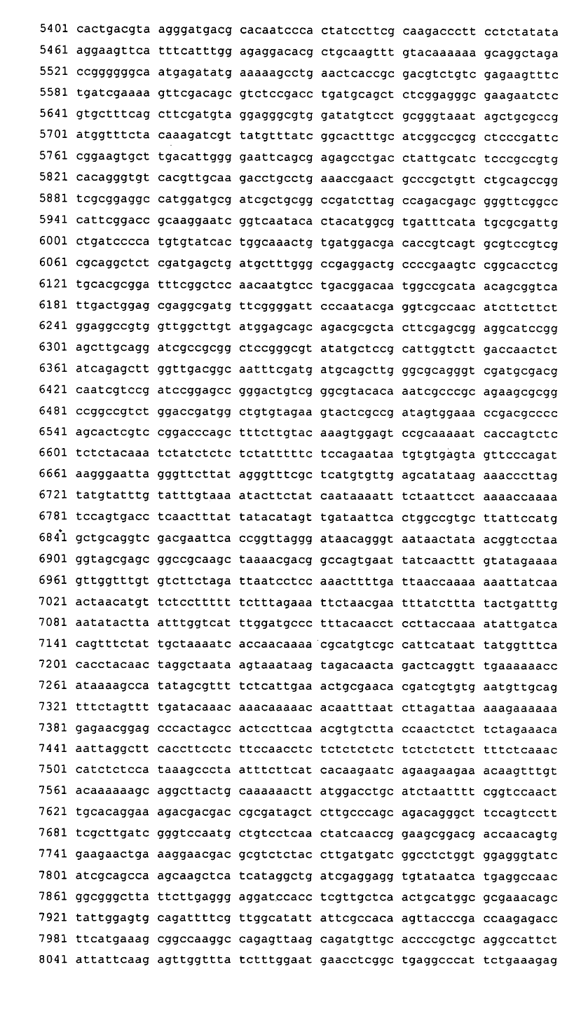

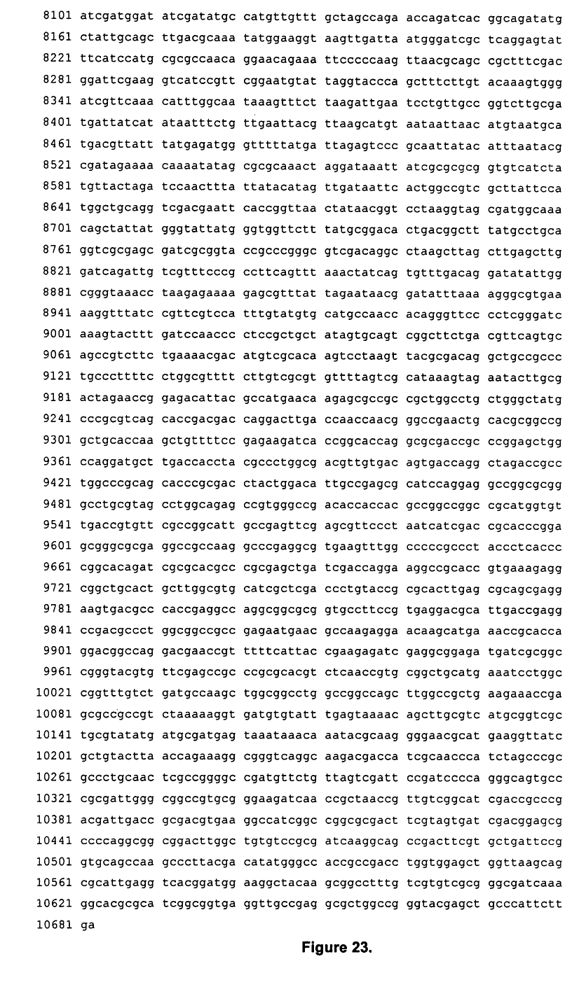

FIG. 23 shows nucleotide sequence of vector pBMVhAtMYB32xs::ipt-nos (Sequence ID No. 14)

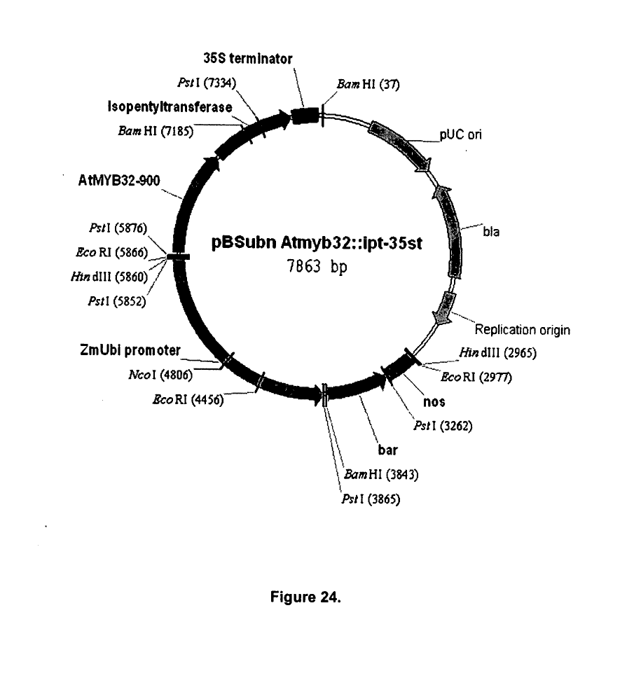

FIG. 24 shows vector details for pBSubn-AtMYB32-900::ipt-nos [Gene: Isopentyl transferase (IPT); Vector: pBSubn-AtMYB32-900::ipt-nos (backbone pPZPRCS2); Selectable marker: spec; Plant selectable marker cassette: Ubi::bar::nos; Gene promoter: AtMYB32-900; Gene terminator: nos.]

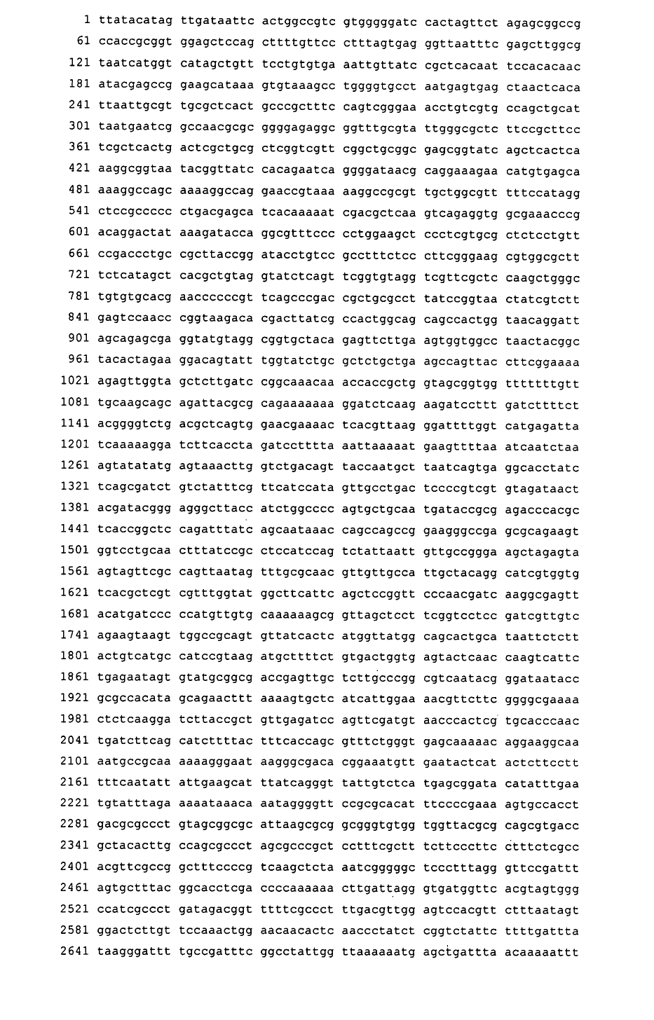

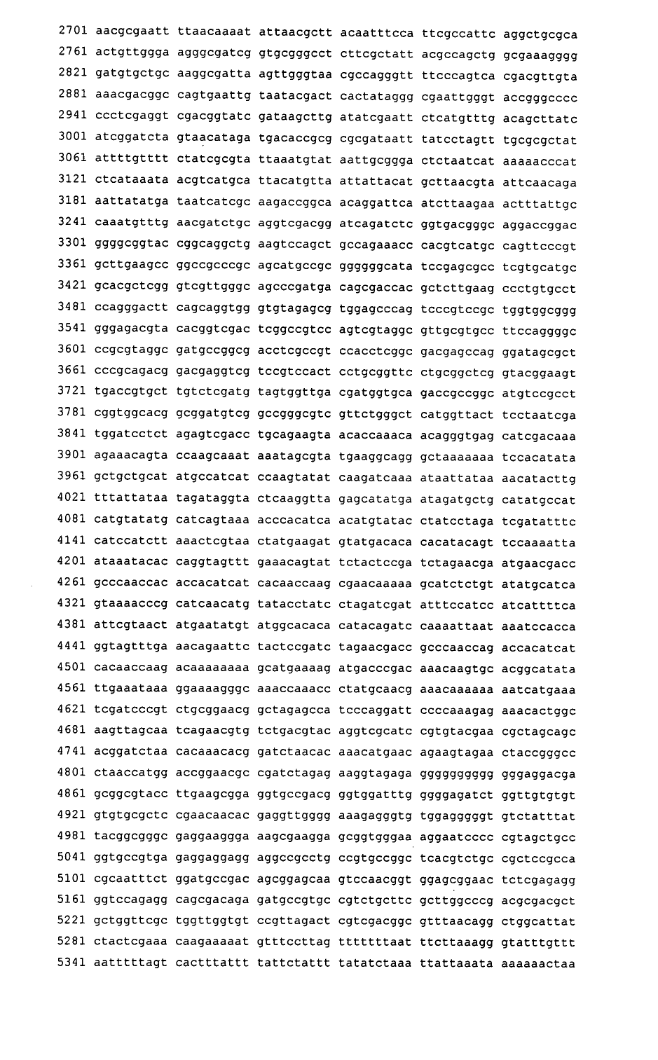

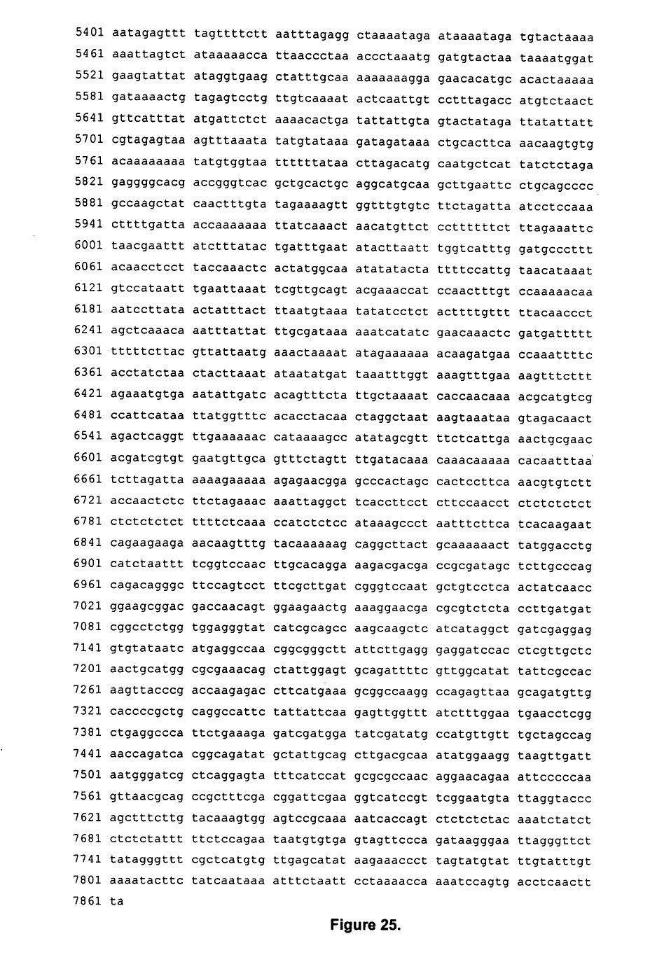

FIG. 25 shows nucleotide sequence of vector pBSAtMYB32900::ipt-nos (Sequence ID No. 15)

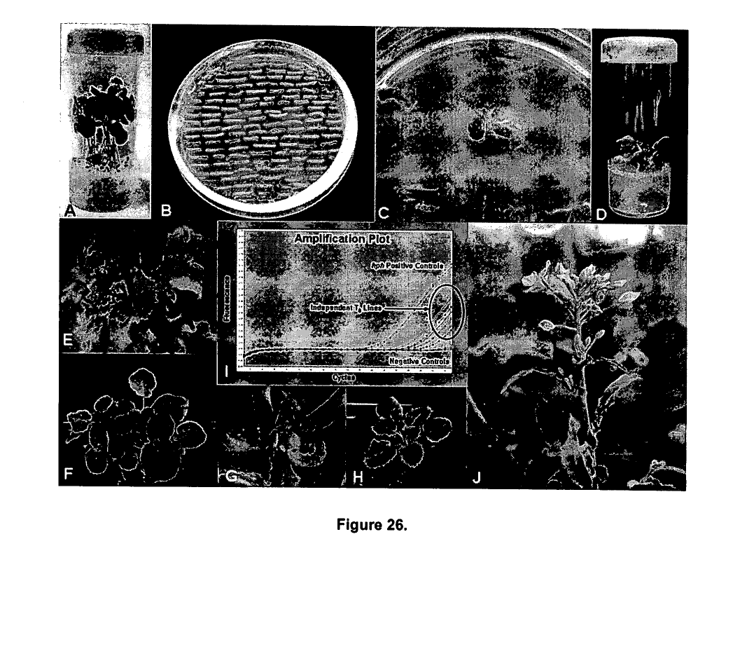

FIG. 26 shows generation of transgenic canola containing the pBMVhATMYB3-900::ipt-nos and pBMVhATMYB32xs::ipt-nos. A. Canola seeds are germinated in vitro; B. Hypocotyl sections are excised from 7-day-old seedlings and inoculated with an Agrobacterium suspension; C&D. Regeneration from inoculated hypocotyl sections under hygromycin selection; E-J. Transgenic T₀ canola plants carrying the pBMVhA TMYB3-900::ipt-nos and pBMVhA TMYB32xs::ipt-nos vectors.

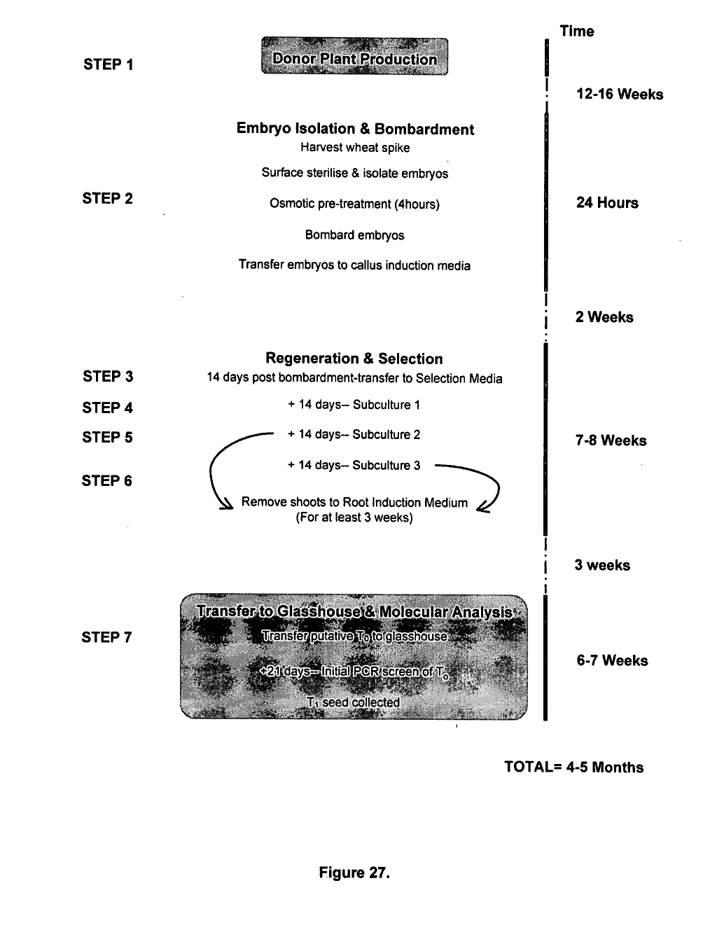

FIG. 27 shows a process for biolistic transformation of wheat.

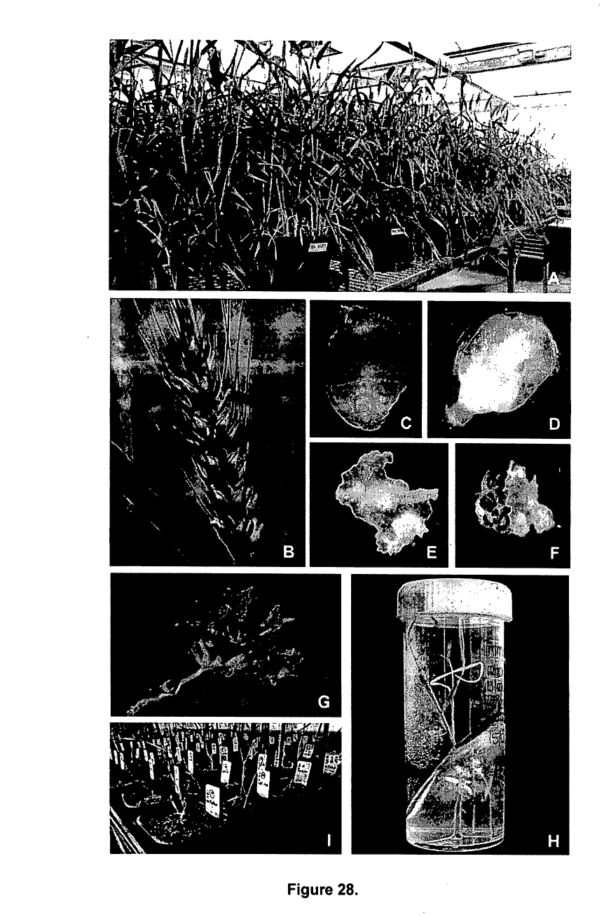

FIG. 28 shows biolistic transformation of wheat (Triticum aestivum L. MPB Bobwhite 26). Donor plant production (A & B); zygotic embryo isolation (C&D); Regeneration under glufosinate selection (E-G); Root formation under selection (H); T₀ plants growing under containment glasshouse conditions for recovery of transgenic offspring (I).

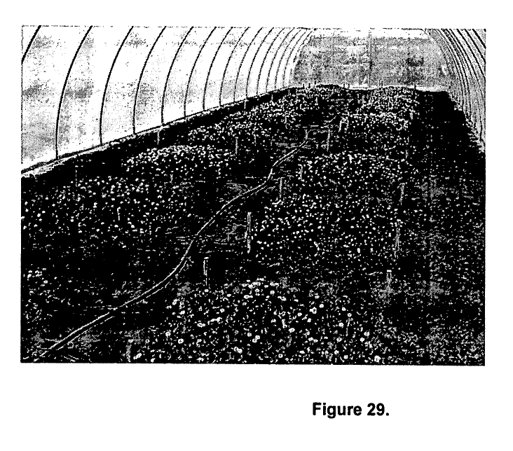

FIG. 29 shows contained field trial of transgenic white clover plants expressing chimeric Atmyb32::ipt genes.

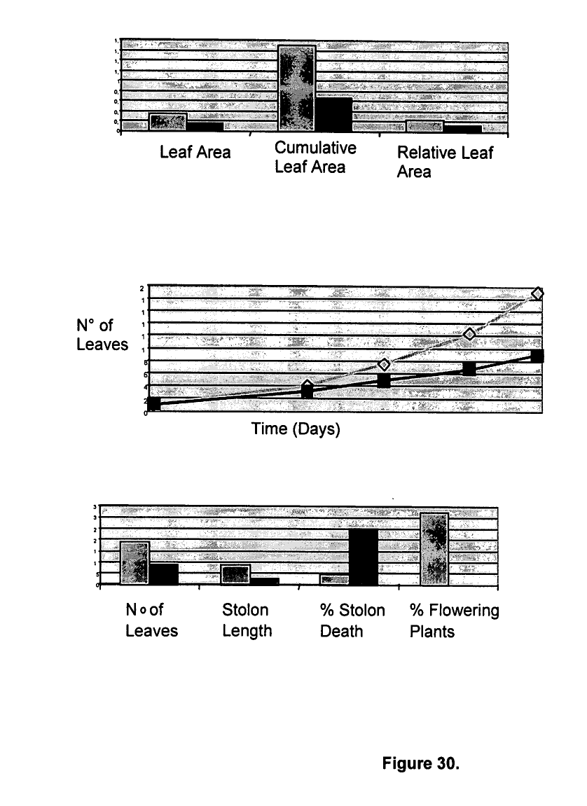

FIG. 30 shows comparative assessment of growth rates and growth dynamics of transgenic white clover plants expressing chimeric Atmyb32::ipt genes with non-transgenic control white clover plants. A) Growth Rates B) Growth Dynamics C) Growth Characteristics (after 45 days)
□ Transgenic white clover ▪ Non-transgenic control white clover (light) (dark)

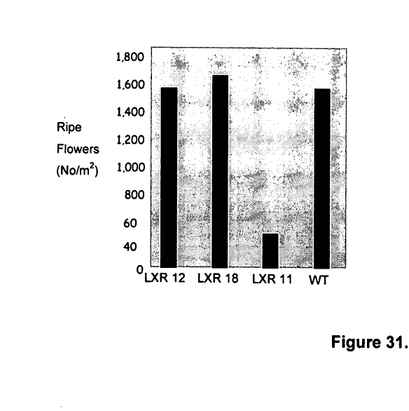

FIG. 31 shows fowering intensity (i.e. number of ripe flower per m²) of transgenic white clover plants expressing chimeric Atmyb32::ipt genes (i.e. LXR 12, LXR 18 and LXR 11) and non-transgenic control white clover plants (i.e. WT) under contained field conditions.

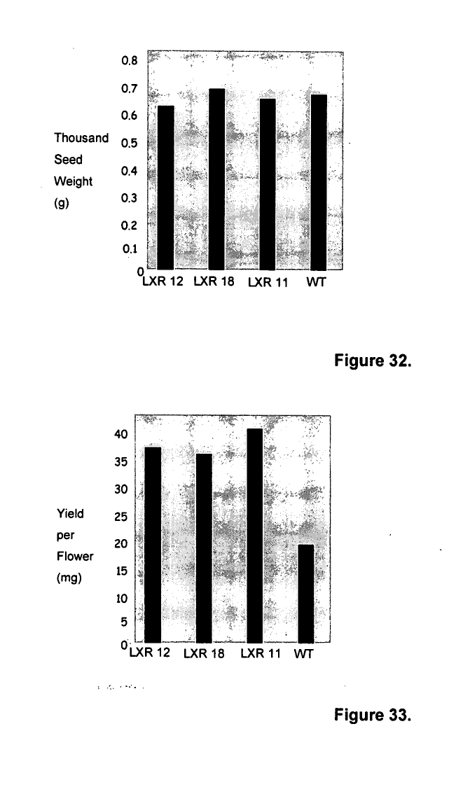

FIG. 32 shows seed weight (i.e. weight of thousand seeds, in grams) of transgenic white clover plants expressing chimeric Atmyb32::ipt genes (i.e. LXR 12, LXR 18 and LXR 11) and non-transgenic control white clover plants (i.e. WT) under contained field conditions.

FIG. 33 shows seed yield per flower (in milligrams) of transgenic white clover plants expressing chimeric Atmyb32::ipt genes (i.e. LXR 12, LXR 18 and LXR 11) and non-transgenic control white clover plants (i.e. WT) under contained field conditions.

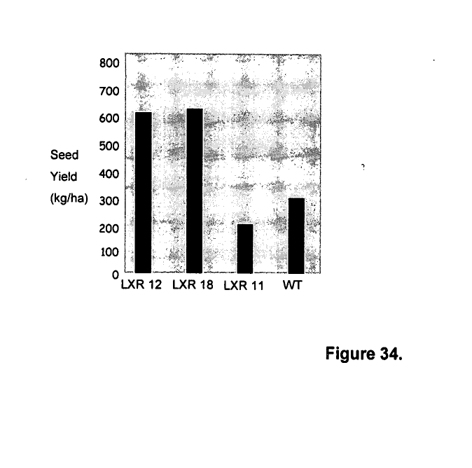

FIG. 34 shows seed yield per area (in kg/ha) of transgenic white clover plants expressing chimeric Atmyb32::ipt genes (i.e. LXR 12, LXR 18 and LXR 11) and non-transgenic control white clover plants (i.e. WT) under contained field conditions.

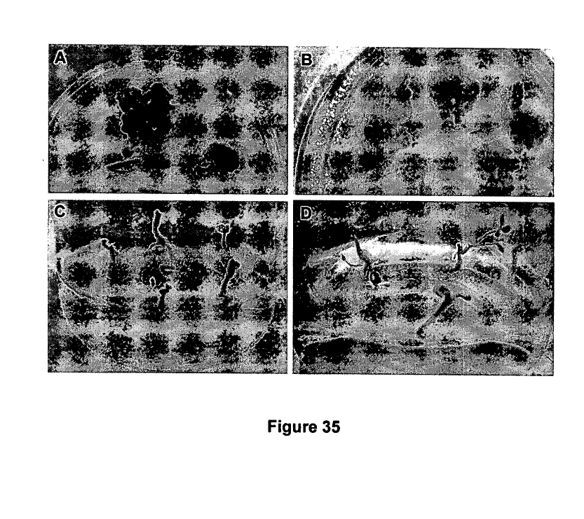

FIG. 35 shows generation of transgenic alfalfa plants containing the chimeric pBMVkATMYB3-900::ipt-nos and pBMVkATMYB32xs::ipt-nos genes A. Petiole explants from alfalfa clones C2-3, C2-4 and 19-17 are used for inoculation with an Agrobacterium suspension and lead to the production of transformed embryogenic calli following selection in presence of kanamycin; B-D. Regeneration of transgenic alfalfa plantlets carrying chimeric genes from pBMVkATMYB3-900::ipt-nos and pBMVkATMYB32xs::ipt-nos vectors, from somatic embryos grown in vitro.

### EXAMPLES

### Example 1

### Atmyb32 Promoter Sequence and Promoter Sequence Variants

The Atmybb32 promoter sequence and variants thereof are shown in FIGS. 1-4.

### Example 2

### Cytokinin Biosynthesis Genes

Examples of cytokinin biosynthesis genes suitable for use in the present invention are shown in FIGS. 6, 8 and 10. Suitable genes also include those encoding the polypeptides shown in FIGS. 7, 9 and 11.

### Example 3

### Production of Transgenic White Clover Plants

Transgenic white clover plants (Trifolium repens cv. Haifa and Irrigation) were produced by Agrobacterium-mediated transformation using a binary vector carrying the chimeric atmyb32::ipt gene (FIG. 12a). The transgenic plants were screened by PCR using ipt and nptII primers (FIG. 12b). HindIII digested genomic DNA samples subjected to Southern DNA hybridization analysis showed that the DNA fragments greater than 4.4 kb were detected in all lanes by both ipt and nptII probes, demonstrating the presence and integration of full-length T-DNA into the white clover genome (FIG. 12). Transgenic lines Hmi01, Imi06, Imi11, and Imi18 (Lane 1, 3, 5, 8 and 12 respectively) appeared to have a single copy of full-length T-DNA integrated in the genome. Other transgenic lines had multiple copies of the atmyb32::ipt transgene.

### Example 4

### Expression of atmyb32::ipt Gene in Transgenic White Clover Plants

The expression of the atmyb32::ipt transgene in transgenic white clover (T repens) plants was assessed by RT-PCR. The ipt mRNA was detected in leaf tissues of all atmyb32::ipt transgenic white clover plants examined, with varying levels of PCR products detected (FIG. 13).

### Example 5

### Delayed Detached Leaf Senescence in Transgenic White Clover Plants

Experiments were performed to assess detached leaf senescence of atmyb32::ipt transgenic plants. Rapid yellowing was observed in detached leaves from non-transformed and atmyb32::gusA transgenic white clover plants of both cultivars within one week. Transgenic lines Hmi01, Hmi08, Imi16 and Imi18 showed delayed senescence while Imi11 and Imi12 showed no sign of yellowing by the end of 7 days. After two weeks, the leaves of all atmyb32::ipt transgenic plants were much greener than those of non-transformed and atmyb32::gusA control transgenic plants (FIG. 14). The degree of senescence in excised leaves was in the order HC, Hmg>Hmi01>Hmi08 for cv. Haifa, and IC and 1 mg>Imi16>Imi18>Imi11 and Imi12 for cv. Irrigation. HC is Haifa untransformed control, Hmg is Haifa atmyb32::gusA control, IC is Irrigation untransformed control, 1 mg is Irrigation atmyb32::gusA control. Hmi01, Hmi08, Imi16, Imi18, Imi11 and Imi12 are independent atmyb32::ipt transgenic white clover plants from the cultivar Haifa (H) and Irrigation (I), respectively.

### Example 6

### Normal Plant Morphology and Root Development in Transgenic White Clover Plants

Normal plant morphology as well as normal shoot and normal root development was observed in atmyb32:ipt transgenic white clover plants (FIG. 6), thus indicating that the regulated expression of the ipt gene under control of the atmyb32 promoter did not negatively affect neither rooting nor apical dominance of the transgenic white clover plants (Table 1).

TABLE 1

| Transformant | Cultivar | Construct | ipt copy No | Phenotype |
|---|---|---|---|---|
| Hmi01 | Haifa | atmyb32::ipt | 1 | Normal |
| Hmi08 | Haifa | atmyb32::ipt | >3 | Normal |
| Imi06 | Irrigation | atmyb32::ipt | 1 | Normal |
| Imi07 | Irrigation | atmyb32::ipt | 3 | Normal |
| Imi09 | Irrigation | atmyb32::ipt | >3 | Normal |
| Imi10 | Irrigation | atmyb32::ipt | >3 | Normal |
| Imi11 | Irrigation | atmyb32::ipt | 1 | Normal |
| Imi12 | Irrigation | atmyb32::ipt | 2 | Normal |
| Imi16 | Irrigation | atmyb32::ipt | 2 | Normal |
| Imi18 | Irrigation | atmyb32::ipt | 1 | Normal |

Normal plant morphology and normal rooting was observed in ten independent atmyb32::ipt transgenic white clover lines analyzed. Estimated ipt gene copy numbers in the ten independent atmyb32::ipt transgenic white clover lines are shown.

### Example 7

### Generation of Vectors for Plant Transformation

Four binary vectors have been generated for Agrobacterium-mediated transformation of plants (FIGS. 16-19). Each vector has a pPZP200 vector backbone (Hajdukiewicz et al., 1994) and contains either chimeric Atmyb32-900::ipt-nos or Atmyb32-xs::ipt-nos with or without a chimeric 35S::nptII-35st or 35S::hph-35st selectable marker cassettes.

One transformation vector has been constructed for biolistic transformation (FIGS. 20 and 21). The transformation vector contains chimeric Atmyb32-900::ipt-35st with a chimeric Ubi::bar-nos selectable marker cassette.

The Atmyb32 promoter, promoter variant Atmyb32xs, the isopentyl transferase gene and terminators 35st and nos were amplified by PCR using Gateway™ (Invitrogen) adapted primers and cloned into a pDONR221 entry vectors. These were subsequently cloned using recombination into destination vectors containing the conventionally cloned selectable marker cassettes. All vectors were fully sequenced following strict quality assurance protocols.

### Example 8

### Agrobacterium-Mediated Transformation of Canola (Brassica napus)

Binary vectors pBMVhATMYB3-900::ipt-nos (FIG. 20) and pBMVhATMYB32xs::ipt-nos (FIG. 22) containing chimeric ipt genes under control of Atmyb32 promoter (FIG. 1) and Atmyb32xs variant promoter sequence with deleted root-specific motifs (FIG. 2) were used for Agrobacterium-mediated transformation of Brassica napus hypocotyl segments (FIG. 26).

Brassica napus seeds are surface sterilised in 70% ethanol for 2 minutes, washed 3 times in sterile water then further surface sterilised in a solution containing 1% (w/v) Calcium hypochlorite and 0.1% (v/v) Tween 20 for 30 minutes. The seeds are washed at least 3 times in sterile water and planted in 120 ml culture vessels containing a solidified germination medium containing 1× Murashige and Skoog (Murashige and Skoog Physiol. Plant, 15: 473-497, 1962) macronutrients, 1× micronutrients and B5 organic vitamins, supplemented with 500 mg/L MES, 2% (w/v) sucrose at a pH of 5.8 with the addition of 4 g/L Gelrite. The vessels are incubated at 25° C. under 16 h light/8 h dark conditions for 7 days to encourage germination.

After 7 days, seedlings of Brassica napus (whole seedlings) are transferred to a liquid medium consisting of 1× Murashige and Skoog macronutrients, 1× micronutrients and B5 organic vitamins, supplemented with 500 mg/L MES, 3% (w/v) sucrose at a pH of 5.8. Seedlings are grouped together and the roots and cotyledons removed prior to cutting the hypocotyls into 7-10 mm sections and plating on 9×1.5 cm petri dishes containing a preconditioning medium consisting of 1× Murashige and Skoog macronutrients, 1× micronutrients and B5 organic vitamins, supplemented with 500 mg/L MES, 3% (w/v) sucrose at a pH of 5.8 solidified with 6.4 g/l Bacto-Agar.

Hypocotyl sections are cultured for 24 hours prior to inoculation with an Agrobacterium suspension OD₆₀₀=0.2 for 30 minutes consisting of 1× Murashige and Skoog macronutrients, 1× micronutrients and B5 organic vitamins, supplemented with 500 mg/L MES, 100 μM Acetosyringone, 3% (w/v) sucrose at a pH of 5.8.

Following inoculation, hypocotyl sections are blotted on sterile paper towels and transferred to 9×1.5 cm petri dishes containing 1× Murashige and Skoog macronutrients, 1× micronutrients and B5 organic vitamins, supplemented with 500 mg/L MES, 100 μM Acetosyringone, 1 mg/L 2,4-D, 3% (w/v) sucrose at a pH of 5.8 solidified with 8 g/l Bacto-Agar. Explants are incubated at 25° C. under 16 h light/8 h dark conditions for 72 hours for co-cultivation.

Following co-cultivation, 20-30 hypocotyl explants are transferred to 9×1.5 cm petri dishes containing a solidified selection medium consisting of 1× Murashige and Skoog macronutrients, 1× micronutrients and B5 organic vitamins, supplemented with 500 mg/L MES, 1 mg/L 2,4-D, 3% (w/v) sucrose at a pH of 5.8 solidified with 8 g/l Bacto-Agar, supplemented with 250 mg/l timentin and 10 mg/l hygromycin to select for hygromycin-resistant shoots. Plates are incubated at 25° C. under 16 h light/8 h dark conditions.

After 7 days hypocotyl explants are transferred to 9×2.0 cm petri dishes containing a solidified regeneration media consisting of 1× Murashige and Skoog macronutrients, 1× micronutrients and B5 organic vitamins, supplemented with 500 mg/L MES, 1 mg/L 2,4-D, 3% (w/v) sucrose at a pH of 5.8 solidified with 8 g/l Bacto-Agar, supplemented with 4 mg/l BAP, 2 mg/l Zeatin, 5 mg/l Silver Nitrate, 250 mg/l timentin and 10 mg/l hygromycin. Plates are incubated under direct light at 25° C. under fluorescent light conditions (16 hr light/8 hr dark photoperiod; 55 μmol m⁻² sec⁻¹) for 4 weeks to encourage shoot development.

Regeneration is monitored weekly and hypocotyl explants transferred to fresh 9×2.0 cm petri dishes containing solidified regeneration media, RM supplemented with 4 mg/l benzyladenine, 2 mg/l zeatin, 5 mg/l silver nitrate, 250 mg/l timentin and 10 mg/l hygromycin for 6-8 weeks to encourage shoot development.

Hygromycin-resistant (Hyg^{r}) shoots are transferred to 120 ml vessels containing solidified root induction medium, RIM1, consisting of 1× Murashige and Skoog macronutrients, 1× micronutrients and B5 organic vitamins, supplemented with 500 mg/L MES, 1 mg/L 2,4-D, 1% (w/v) sucrose at a pH of 5.8 solidified with 8 g/l Bacto-Agar supplemented with 250 mg/l timentin. Shoots are incubated under direct fluorescent light at 25° C. (16 hr light/8 hr dark photoperiod; 55 μmol m⁻² sec⁻¹) to encourage shoot elongation and root development over 4-5 weeks. All Hyg^{r} shoots with developed shoot and root systems are transferred to soil and grown under glasshouse conditions.

### Example 9

### Biolistic Transformation of Wheat (Triticum aestivum L.)

Transformation vectors containing chimeric ipt genes under control of Atmyb32 promoter (FIG. 1) and Atmyb32xs variant promoter sequence with deleted root-specific motifs (FIG. 2) were used for biolistic transformation of wheat (Triticum aestivum L. MPB Bobwhite 26). A representative vector is shown in FIG. 24. A schematic of the procedure for biolistic transformation of wheat is outlined in FIG. 27. The transformation procedure includes the following steps:

Step 1 (Donor Plant Production):

Triticum aestivum (Bobwhite 26) seed is used for the production of donor plant material. Wheat plants are grown in a nursery mix consisting of composted pine bark, perlite and vermiculite, with five plants per pot to a maximum pot size of 20 cm. Plants are kept under glasshouse conditions at approximately 22-24° C. for 12-16 weeks (FIG. 28A). Once the first spike emerges from the flag leaf, plants are tagged and embryos collected from the tallest heads 12-15 days post anthesis.

Step 2(Day1)

Spikes at the desired stage of development are harvested (FIG. 28B). Caryopsis are removed from the spikes and surface sterilised for 20 minutes in a 0.8% (v/v) NaOCl solution and rinsed at least four times in sterile distilled water.

Embryos up to 10 mm in length are aseptically excised from each caryopsis (removing the axis) using a dissecting microscope and cultured axial side down on an osmotic medium (E3maltose) consisting of 2× Murashige and Skoog (1962) macronutrients, 1× micronutrients and organic vitamins, 40 mg/L thiamine, 150 mg/L L-asparagine, supplemented with 15% (w/v) maltose, 0.8% (w/v) Sigma-agar and 2.5 mg/L 2,4-D (FIGS. 28C&D). Embryos are cultured on 60 mm×15 mm clear polypropylene petrie dishes with 15 mL of media. Culture plates are incubated at 24° C. in the dark for 4 hours prior to bombardment. Embryos are bombarded using a BioRad PDS1000 gene gun at 900 psi and at 6 cm with 1 μg of vector plasmid DNA precipitated onto 0.6 μm gold particles. Following bombardment, embryos are incubated overnight in the dark on the osmotic media.

Step 3 (Day 2):

Embryos are transferred to a callus induction medium (E3calli) consisting of 2× Murashige and Skoog (1962) macronutrients and 1× micronutrients and organic vitamins, 40 mg/L thiamine, 150 mg/L L-asparagine, supplemented with 6% (w/v) sucrose, 0.8% (w/v) Sigma-agar and 2.5 mg/L 2,4-D. Embryos are cultured for two weeks at 24° C. in the dark.

Step 4 (Day 16):

After 2 weeks of culture on E3calli, embryos have produced embryogenic callus and are subcultured onto a selection medium (E3Select) consisting of 2× Murashige and Skoog (1962) macronutrients and 1× micronutrients and organic vitamins, 40 mg/L thiamine, 150 mg/L L-asparagine, supplemented with 2% (w/v) sucrose, 0.8% (w/v) Sigma-agar, 5 mg/L of D,L phosphinothricin (PPT) and no plant growth regulators (FIG. 28E-G). Cultures are incubated for further 14 days on E3Select at 24° C. in the light and a 12-hour photoperiod.

Step 5 (Day 30):

After 14 days culture on E3Select, embryogenic callus is sub-cultured onto fresh E3Select for a further 14 days (FIG. 28E-G).

Step 6 (Day 44):

After about 4 weeks on E3Select, developing plantlets are excised from the embryonic callus mass and grown for a further three weeks in 65 mm×80 mm or 65 mm×150 mm polycarbonate tissue culture vessels containing root induction medium (RM). Root induction medium consists of 1× Murashige and Skoog (1962) macronutrients, micronutrients and organic vitamins, 40 mg/L thiamine, 150 mg/L L-asparagine, supplemented with 2% (w/v) sucrose, 0.8% (w/v) Sigma-agar, and 5 mg/L of PPT (FIG. 28H). Remaining embryogenic callus is sub-cultured onto E3Select for another 14 days.

Step 7 (Day 65+):

Regenerated plantlets surviving greater than 3 weeks on RM with healthy root formation are potted into a nursery mix consisting of peat and sand (1:1) and kept at 22-24° C. with elevated humidity under a nursery humidity chamber system (FIG. 28). After two weeks, plants are removed from the humidity chamber and hand watered and liquid fed Aquasol™ weekly until maturity. The T₀ plants are sampled for genomic DNA and molecular analysis. T₁ seed is collected and planted for high-throughput Q-PCR analysis (FIG. 28J).

### Example 10

### Agronomic Performance of Transgenic White Clover Plants

The agronomic performance of atmyb32::ipt transgenic white clover (Trifolium repens) plants, relative to that of non-transgenic control white clover plants, was evaluated under environmentally controlled growth chamber conditions and in contained field trials (FIG. 29).

Transgenic white clover plants expressing chimeric Atmyb32::ipt genes assessed under controlled growth chamber conditions revealed a significantly enhanced biomass accumulation and reductions in manifestations of senescence, when compared with non-transgenic control white clover plants (FIG. 30). The transgenic white clover plants expressing chimeric Atmyb32::ipt genes showed enhanced total leaf area, increased cumulative leaf area, higher leaf growth dynamics (i.e. number of leaves over time), higher stolon length and increased % flowering plants as well as reduced stolon senescence and death compared with non-transgenic control white clover plants (FIG. 30A-C).

The seed yield performance of 3 independent atmyb32::ipt expressing transgenic white clover plants (i.e. LXR 12, LXR 18 and LXR 11) was also comparatively assessed with non-transgenic control plants (i.e. wild type, WT) under contained field conditions. Two independent atmyb32::ipt expressing transgenic white clover plants (i.e. LXR 12 and LXR 18) with indistinguishable flowering intensity (i.e. number of ripe flowers per m²) to the non-transgenic control plant (i.e. WT) were selected for field evaluation (FIG. 31).

While the seed weight (i.e. weight of thousand seeds) of transgenic white clover plants expressing chimeric Atmyb32::ipt genes (i.e. LXR 12, LXR 18 and LXR 11) was indistinguishable from non-transgenic control white clover plants (i.e. WT) (FIG. 32), the total seed yield expressed on the basis of per flower (FIG. 33), and per area sown (FIG. 34) was doubled in transgenic white clover plants expressing chimeric Atmyb32::ipt genes (i.e. LXR 12 and LXR 18) when compared with non-transgenic control white clover plants of equivalent flowering intensity (i.e. WT).

### Example 11

### Agrobacterium-Mediated Transformation of Alfalfa (Medicago sativa)

The binary vector pBMVkATMYB32xs::ipt-nos (FIG. 18) containing chimeric ipt genes under control of Atmyb32xs variant promoter sequence with deleted root-specific motifs (FIG. 2) was used for Agrobacterium-mediated transformation of Medicago sativa petiole explants from highly-regenerable alfalfa (M. sativa) clones C2-3, C2-4 and 19-17 (FIG. 35).

Following co-cultivation with Agrobacterium tumefaciens strain LBA 4404 harbouring the binary vector pBMVkATMYB32xs::ipt-nos, the alfalfa explants were washed with medium containing cefotaxime and used for induction of embryogenic callus under selective medium containing 25 mg/l kanamycin. Transgenic embryogenic alfalfa calli were recovered and allowed to regenerate transgenic alfalfa shoots, which were transferred on rooting medium leading to the recovery of transgenic alfalfa plants expressing chimeric ipt genes under control of Atmyb32xs variant promoter (FIG. 35).

It will be understood that the invention disclosed and defined in this specification extends to all alternative combinations of two or more of the individual features mentioned or evident from the text or drawings. All of these different combinations constitute various alternative aspects of the invention.

It will also be understood that the term “comprises” (or its grammatical variants) as used in this specification is equivalent to the term “includes” and should not be taken as excluding the presence of other elements or features.

Documents cited in this specification are for reference purposes only and their inclusion is not an acknowledgement that they form part of the common general knowledge in the relevant art.

Finally, it is to be understood that various alterations, modifications and/or additions may be made without departing from the spirit of the present invention as outlined herein.

1. A method of manipulating senescence in a plant, said method including introducing into said plant a genetic construct including a modified myb gene promoter, or a functionally active fragment or variant thereof, operatively linked to a gene encoding an enzyme involved in biosynthesis of a cytokinin, or a functionally active fragment or variant thereof, wherein one or more root specific motifs and/or pollen specific motifs are deleted or inactivated in said modified myb gene promoter.

2. A method according to claim 1 wherein one or more root specific motifs are deleted or inactivated and wherein each of said root specific motifs includes a consensus sequence ATATT or AATAT.

3. A method according to claim 1 wherein one or more pollen specific motifs are deleted or inactivated and wherein each of said pollen specific motifs includes a consensus sequence TTCT or AGAA.

4. A method according to claim 1 wherein said myb gene promoter is from Arabidopsis.

5. A method according to claim 1 wherein said myb gene promoter includes a nucleotide sequence selected from the group consisting of SEQ ID Nos: 2, 3 and 4, and functionally active fragments and variants thereof.

6. A method according to claim 1 wherein said gene encoding an enzyme involved in biosynthesis of a cytokinin is an isopentenyl transferase (ipt) gene or a functionally active fragment or variant thereof.

7. A method according to claim 6 wherein said gene encoding an enzyme involved in biosynthesis of a cytokinin is from a genus selected from the group consisting of Agrobacterium, Lotus and Petunia.

8. A method according to claim 7 wherein said gene encoding an enzyme involved in biosynthesis of a cytokinin includes a nucleotide sequence selected from the group consisting of Sequence ID Nos: 5, 7 and 9, sequences encoding the polypeptides having a sequence of SEQ ID Nos: 6, 8 or 10, and functionally active fragments and variants thereof.

9. A method according to claim 1 wherein said genetic construct is introduced into said plant by Agrobacterium-mediated or biolistic transformation of plant cells.

10. A method according to claim 9 wherein plant cells incorporating the genetic construct are selected and then cultured to regenerate transformed plants.

11. A vector capable of manipulating senescence in a plant, said vector including a modified myb gene promoter, or a functionally active fragment or variant thereof, operatively linked to a gene encoding an enzyme involved in the biosynthesis of a cytokinin, or a functionally active fragment or variant thereof, wherein one or more root specific motifs and/or pollen specific motifs are deleted or inactivated in said modified myb gene promoter.

12. A vector according to claim 11, further including a terminator; said promoter, gene and terminator being operatively linked.

13. A vector according to claim 11 wherein said one or more root specific motifs are deleted or inactivated in said modified myb gene promoter and wherein each of said root specific motifs includes a consensus sequence ATATT or AATAT.

14. A vector according to claim 11 wherein one or more pollen specific motifs are deleted or inactivated in said modified myb promoter and wherein each of said pollen specific motifs includes a consensus sequence TTCT or AGAA.

15. A vector according to claim 11 wherein said myb gene promoter is from Arabidopsis.

16. A vector according to claim 11 wherein said myb gene promoter includes a nucleotide sequence selected from the group consisting of Sequence ID Nos: 2, 3 and 4, and functionally active fragments and variants thereof.

17. A vector according to claim 11 wherein said gene encoding an enzyme involved in biosynthesis of a cytokinin is an isopentenyl transferase (ipt) gene or a functionally active fragment or variant thereof.

18. A vector according to claim 17 wherein said gene encoding an enzyme involved in biosynthesis of a cytokinin is from a genus selected from the group consisting of Agrobacterium, Lotus and Petunia.

19. A vector according to claim 17 wherein said gene encoding an enzyme involved in biosynthesis of a cytokinin includes a nucleotide sequence selected from the group consisting of SEQ ID Nos: 5, 7 and 9, and sequences encoding polypeptides having a sequence of SEQ ID Nos: 6, 8 or 10.

20. A transgenic plant cell, plant, plant seed or other plant part, with modified senescence characteristics, said plant cell, plant, plant seed or other plant part including a genetic construct including a modified myb gene promoter, or a functionally active fragment or variant thereof, operatively linked to a gene encoding an enzyme involved in biosynthesis of a cytokinin, or a functionally active fragment or variant thereof, wherein one or more root specific motifs and/or pollen specific motifs are deleted or inactivated in said modified myb gene promoter.

21. A transgenic plant cell, plant, plant seed or other plant part, according to claim 20 produced by a method including introducing into said plant a genetic construct including a modified myb gene promoter, or a functionally active fragment or variant thereof, operatively linked to a gene encoding an enzyme involved in biosynthesis of a cytokinin, or a functionally active fragment or variant thereof, wherein one or more root specific motifs and/or pollen specific motifs are deleted or inactivated in said modified myb gene promoter.

22. A transgenic plant, plant seed or other plant part derived from a plant cell according to claim 20.

23. A transgenic plant, plant seed or other plant part derived from a plant according to claim 20.

24. A method of manipulating senescence in a plant, said method including introducing into said plant a genetic construct including a modified myb gene promoter, having between one and ten root specific motifs deleted or inactivated, said root specific motifs having a consensus sequence ATATT or AATAT, or having between one and thirty pollen specific motifs deleted or inactivated, said pollen specific motifs having a consensus sequence TTCT or AGAA; said modified myb gene promoter being operatively linked to an ipt or sho gene.

25. A method of enhancing biomass in a plant, said method include introducing into said plant a genetic construct including a myb gene promoter, or a functionally active fragment or variant thereof, operatively linked to a gene encoding an enzyme involved in biosynthesis of a cytokinin, or a functionally active fragment or variant thereof.

26. a vector capable of enhancing biomass in a plant, said vector including a myb gene promoter, or a functionally active fragment or variant thereof, operatively linked to a gene encoding an enzyme involved in the biosynthesis of a cytokinin, or a functionally active fragment or variant thereof.


SEQUENCE LISTING (15 sequences)

SEQ ID NO: 1 - DNA, 941 bp, Arabidopsis thaliana
gtttgtgtcttctagattaatcctccaaacttttgattaaccaaaaaaattatcaaactaacatgttctccttttttctttagaaattctaacgaatttatctttatactgatttgaatatacttaatttggtcatttggatgccctttacaacctccttaccaaactcactatggcaaatatatactattttccattgtaacataaatgtccataatttgaattaaattcgttgcagtacgaaaccatccaactttgtccaaaaacaaaatccttataactatttactttaatgtaaatatatcctctacttttgtttttacaaccctagctcaaacaaatttattatttgcgataaaaaatcatatcgaacaaactcgatgattttttttttcttacgttattaatgaaactaaaatatagaaaaaaacaagatgaaccaaattttcacctatctaactacttaaatataatatgattaaatttggtaaagtttgaaaagtttctttagaaatgtgaaatattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaa

SEQ ID NO: 2 - DNA, 588 bp, Arabidopsis thaliana
gtttgtgtcttctagattaatcctccaaacttttgattaaccaaaaaaattatcaaactaacatgttctccttttttctttagaaattctaacgaatttatctttatactgatttgaatatacttaatttggtcatttggatgccctttacaacctccttaccaaactcattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaa

SEQ ID NO: 3 - DNA, 468 bp, Arabidopsis thaliana
tacttaatttggtcatttggatgccctttacaacctccttaccaaactcattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaa

SEQ ID NO: 4 - DNA, 419 bp, Arabidopsis thaliana
attgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaa

SEQ ID NO: 5 - DNA, 723 bp, Agrobacterium tumefaciens
atggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattag

SEQ ID NO: 6 - protein, 240 aa, Agrobacterium tumefaciens
MDLHLIFGPTCTGKTTTAIALAQQTGLPVLSLDRVQCCPQLSTGSGRPTVEELKGTTRLYLDDRPLVEGIIAAKQAHHRLIEEVYNHEANGGLILEGGSTSLLNCMARNSYWSADFRWHIIRHKLPDQETFMKAAKARVKQMLHPAAGHSIIQELVYLWNEPRLRPILKEIDGYRYAMLFASQNQITADMLLQLDANMEGKLINGIAQEYFIHARQQEQKFPQVNAAAFDGFEGHPFGMY

SEQ ID NO: 7 - DNA, 975 bp, Lotus japonicus
atgtccatctcaatgctaatgtgcagactaagacaacccttaataaacgttccctgcagtggcaaaaaactgagcatgaggcagattcaaaaggagaaggtagtgttggtgatgggagctacagggacaggaaagtcaaagctctccattgacctcgccacctgtttcccctcagaaatcatcaactccgacaagattcaaatctacgacggcctcgacatcgtcaccaacaaaatctccaaggaagaacaacgtggaatcccccaccacctcctcggaactcaaaaccctaacacagacttcaccgccggcgatttcagtgactgttccaccgccgccattgacgcaatcacaagccgcgaccaccttccgatcatcgccggaggttcgaactcctacctggaggcgttaatcgacgacgacgactacaaattccgatcgaggtacgacttctgctgcctctgggtcgacgtggcaatgccggtgctggactcatacgtggcggcgcgtgtggatcagatgctccggagcggaatggtggaggagctgagaccgtttttcaacgcgaacggcgactactcgagaggaatcagaagagcgattggggttcctgaattcgacgagtatttccggcgggaagggttcgccgatgaggaaacgaggaaattgttactggagcgagcggtgagggagatgaaggtgaacacgtgcaagctcgcgaggaggcaattggggaagattcagaggctgaggaatgtgaagaggtgggagattcaccgtgttgatgcgacgccggtgttttggaagcgtggggaggaggctgatgaggcgtggcggaaggtggtggcagagcctagtgctatgatcgtagcgcagtttctgtataaggcaaagagtgatgtgaatgttgtttctggcggtttcagagtgccggcgggttcaacggagagtgttatggcggcggcgacgtgttag

SEQ ID NO: 8 - protein, 324 aa, Lotus japonicus
MSISMLMCRLRQPLINVPCSGKKLSMRQIQKEKVVLVMGATGTGKSKLSIDLATCFPSEIINSDKIQIYDGLDIVTNKISKEEQRGIPHHLLGTQNPNTDFTAGDFSDCSTAAIDAITSRDHLPIIAGGSNSYLEALIDDDDYKFRSRYDFCCLWVDVAMPVLDSYVAARVDQMLRSGMVEELRPFFNANGDYSRGIRRAIGVPEFDEYFRREGFADEETRKLLLERAVREMKVNTCKLARRQLGKIQRLRNVKRWEIHRVDATPVFWKRGEEADEAWRKVVAEPSAMIVAQFLYKAKSDVNVVSGGFRVPAGSTESVMAAATC

SEQ ID NO: 9 - DNA, 1053 bp, Petunia x hybrida
atgttaattgtagtacatattattagcatcacacgcatcatattcatcaccttaacccataatcatctccatttccttatgtttagatcattatcatacaatcacaagcacctcaaattccttacaaacccgaccacacgggtactccgaagaaacatgtcgtcatccactgtagtaacaatacccggccccacacaaaaaaacaaaaacaaaatcatagtaataatgggtgcaacaggttcaggaaaatcaaaactctcaatagacctcgtcacacgtcactatcctttttccgaaatcattaactccgacaaaatccaaattaccaaaggtttaaacataaccacaaacaaaatcactgtacccgaccgacgtggcgtagttcatcatttactcggcgagattgaccccgactttaacttttctccttctcatttccggtcaattgctggtcaacgcattaactccattattaatcgccataaactcccattcctcgttggtgggtccaactcatatatctacgctttattaacaaaccggttcgacccggattttaaccctgattcaaacccggttcattttatatccaacgagttacgctacaactgttgttttatttgggtcgatgtattaaacccggttttgaatgagtatttggataaacgggtcgatgagatgatgaactcgggtatgtatgaagaactggaacagttttttaaagaaaacaggttttcggatccgggtttggaaccgggtcgggccaccgggttgaggaaagcgataggggtaccggaaatggagaggtattttaagaagagctgtacgtatgaggaagcagtgagggaaataaaagaaaacacgtggcggttagcgaagaagcagatgtggaagatccaacggttgagagaagcagggtgggacctacaaagagtagatgccacggaggcatttgtggaggcgatgagtaataagaaggaaaagggaattatttgggaaaaacaagtagtggaaccaagtgtcaagattgtgaaccgttttttgttggactga

SEQ ID NO: 10 - protein, 350 aa, Petunia x hybrida
MLIVVHIISITRIIFITLTHNHLHFLMFRSLSYNHKHLKFLTNPTTRVLRRNMSSSTVVTIPGPTQKNKNKIIVIMGATGSGKSKLSIDLVTRHYPFSEIINSDKIQITKGLNITTNKITVPDRRGVVHHLLGEIDPDFNFSPSHFRSIAGQRINSIINRHKLPFLVGGSNSYIYALLTNRFDPDFNPDSNPVHFISNELRYNCCFIWVDVLNPVLNEYLDKRVDEMMNSGMYEELEQFFKENRFSDPGLEPGRATGLRKAIGVPEMERYFKKSCTYEEAVREIKENTWRLAKKQMWKIQRLREAGWDLQRVDATEAFVEAMSNKKEKGIIWEKQVVEPSVKIVNRFLLD

SEQ ID NO: 11 - DNA, 10786 bp, Synthetic/artificial
gtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcctcgagtacgtaggatccatttaaattctagaggcgcgccgatatcctctcttaaggtagcgagctcttaattaatagggataacagggtaatgcggccgcaagctaaaacgacggccagtgaattatcaactttgtatagaaaagttgctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgcaagtttgtacaaaaaagcaggctatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaactttattatacatagttgataattcactggccgtgcttattccatggctgcaggtcgacgaattcaccggttagggataacagggtaatcgctaccttaggaccgttatagttacggccagtgccattaccctgttatccctaaccggtgacaactttgtatagaaaagttggtttgtgtcttctagattaatcctccaaacttttgattaaccaaaaaaattatcaaactaacatgttctccttttttctttagaaattctaacgaatttatctttatactgatttgaatatacttaatttggtcatttggatgccctttacaacctccttaccaaactcactatggcaaatatatactattttccattgtaacataaatgtccataatttgaattaaattcgttgcagtacgaaaccatccaactttgtccaaaaacaaaatccttataactatttactttaatgtaaatatatcctctacttttgtttttacaaccctagctcaaacaaatttattatttgcgataaaaaatcatatcgaacaaactcgatgattttttttttcttacgttattaatgaaactaaaatatagaaaaaaacaagatgaaccaaattttcacctatctaactacttaaatataatatgattaaatttggtaaagtttgaaaagtttctttagaaatgtgaaatattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaacaagtttgtacaaaaaagcaggcttactgcaaaaaacttatggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggtacccagctttcttgtacaaagtgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccaactttattatacatagttgattcgtcgacctgcagtcgctaccttaggaccgttatagttatggcaaacagctattatgggtattatgggtggttctttatgcggacactgacggctttatgcctgcaggtcgcgagcgatcgcggtaccgcccgggcgtcgacaggcctaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttga

SEQ ID NO: 12 - DNA, 10468 bp, Synthetic/artificial
gtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcctcgagtacgtaggatccatttaaattctagaggcgcgccgatatcctctcttaaggtagcgagctcttaattaatagggataacagggtaatgcggccgcaagctaaaacgacggccagtgaattatcaactttgtatagaaaagttgctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgcaagtttgtacaaaaaagcaggctatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaactttattatacatagttgataattcactggccgtgcttattccatggctgcaggtcgacgaattcaccggttagggataacagggtaataactataacggtcctaaggtagcgagcggccgcaagctaaaacgacggccagtgaattatcaactttgtatagaaaagttggtttgtgtcttctagattaatcctccaaacttttgattaaccaaaaaaattatcaaactaacatgttctccttttttctttagaaattctaacgaatttatctttatactgatttgaatatacttaatttggtcatttggatgccctttacaacctccttaccaaaatattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctctttctcaaaccatctctccataaagccctaatttcttcatcacaagaatcagaagaagaaacaagtttgtacaaaaaagcaggcttactgcaaaaaacttatggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggtacccagctttcttgtacaaagtgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccaactttattatacatagttgataattcactggccgtcgcttattccatggctgcaggtcgacgaattcaccggttaactataacggtcctaaggtagcgatggcaaacagctattatgggtattatgggtggttctttatgcggacactgacggctttatgcctgcaggtcgcgagcgatcgcggtaccgcccgggcgtcgacaggcctaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttga

SEQ ID NO: 13 - DNA, 11000 bp, Synthetic/artificial
gtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcctcgagtacgtaggatccatttaaattctagaggcgcgccgatatcctctcttaaggtagcgagctcttaattaatagggataacagggtaatgcggccgcaagctaaaacgacggccagtgaattatcaactttgtatagaaaagttgctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgcaagtttgtacaaaaaagcaggctagaccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccggccgtctggaccgatggctgtgtagaagtactcgccgatagtggaaaccgacgccccagcactcgtccggacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaactttattatacatagttgataattcactggccgtgcttattccatggctgcaggtcgacgaattcaccggttagggataacagggtaatcgctaccttaggaccgttatagttacggccagtgccattaccctgttatccctaaccggtgacaactttgtatagaaaagttggtttgtgtcttctagattaatcctccaaacttttgattaaccaaaaaaattatcaaactaacatgttctccttttttctttagaaattctaacgaatttatctttatactgatttgaatatacttaatttggtcatttggatgccctttacaacctccttaccaaactcactatggcaaatatatactattttccattgtaacataaatgtccataatttgaattaaattcgttgcagtacgaaaccatccaactttgtccaaaaacaaaatccttataactatttactttaatgtaaatatatcctctacttttgtttttacaaccctagctcaaacaaatttattatttgcgataaaaaatcatatcgaacaaactcgatgattttttttttcttacgttattaatgaaactaaaatatagaaaaaaacaagatgaaccaaattttcacctatctaactacttaaatataatatgattaaatttggtaaagtttgaaaagtttctttagaaatgtgaaatattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaacaagtttgtacaaaaaagcaggcttactgcaaaaaacttatggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggtacccagctttcttgtacaaagtgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccaactttattatacatagttgattcgtcgacctgcagtcgctaccttaggaccgttatagttatggcaaacagctattatgggtattatgggtggttctttatgcggacactgacggctttatgcctgcaggtcgcgagcgatcgcggtaccgcccgggcgtcgacaggcctaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttga

SEQ ID NO: 14 - DNA, 10682 bp, Synthetic/artificial
gtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaatcgcttgagttaacgccggcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacatgtcataacaagaagccatgaaaaccgccactgcgccgttaccaccgctgcgttcggtcaaggttctggaccagttgcgtgacggcagttacgctacttgcattacagcttacgaaccgaacgaggcttatgtccactgggttcgtgcccgaattgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattgaattcctcgagtacgtaggatccatttaaattctagaggcgcgccgatatcctctcttaaggtagcgagctcttaattaatagggataacagggtaatgcggccgcaagctaaaacgacggccagtgaattatcaactttgtatagaaaagttgctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgcaagtttgtacaaaaaagcaggctagaccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccggccgtctggaccgatggctgtgtagaagtactcgccgatagtggaaaccgacgccccagcactcgtccggacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaactttattatacatagttgataattcactggccgtgcttattccatggctgcaggtcgacgaattcaccggttagggataacagggtaataactataacggtcctaaggtagcgagcggccgcaagctaaaacgacggccagtgaattatcaactttgtatagaaaagttggtttgtgtcttctagattaatcctccaaacttttgattaaccaaaaaaattatcaaactaacatgttctccttttttctttagaaattctaacgaatttatctttatactgatttgaatatacttaatttggtcatttggatgccctttacaacctccttaccaaaatattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaacaagtttgtacaaaaaagcaggcttactgcaaaaaacttatggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggtacccagctttcttgtacaaagtgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatccaactttattatacatagttgataattcactggccgtcgcttattccatggctgcaggtcgacgaattcaccggttaactataacggtcctaaggtagcgatggcaaacagctattatgggtattatgggtggttctttatgcggacactgacggctttatgcctgcaggtcgcgagcgatcgcggtaccgcccgggcgtcgacaggcctaagcttagcttgagcttggatcagattgtcgtttcccgccttcagtttaaactatcagtgtttgacaggatatattggcgggtaaacctaagagaaaagagcgtttattagaataacggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttga

SEQ ID NO: 15 - DNA, 7862 bp, Synthetic/artificial
ttatacatagttgataattcactggccgtcgtgggggatccactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtaccgggccccccctcgaggtcgacggtatcgataagcttgatatcgaattctcatgtttgacagcttatcatcggatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatctgcaggtcgacggatcagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatggttacttcctaatcgatggatcctctagagtcgacctgcagaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcattttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatctggttgtgtgtgtgtgcgctccgaacaacacgaggttggggaaagagggtgtggagggggtgtctatttattacggcgggcgaggaagggaaagcgaaggagcggtgggaaaggaatcccccgtagctgccggtgccgtgagaggaggaggaggccgcctgccgtgccggctcacgtctgccgctccgccacgcaatttctggatgccgacagcggagcaagtccaacggtggagcggaactctcgagaggggtccagaggcagcgacagagatgccgtgccgtctgcttcgcttggcccgacgcgacgctgctggttcgctggttggtgtccgttagactcgtcgacggcgtttaacaggctggcattatctactcgaaacaagaaaaatgtttccttagtttttttaatttcttaaagggtatttgtttaatttttagtcactttattttattctattttatatctaaattattaaataaaaaaactaaaatagagttttagttttcttaatttagaggctaaaatagaataaaatagatgtactaaaaaaattagtctataaaaaccattaaccctaaaccctaaatggatgtactaataaaatggatgaagtattatataggtgaagctatttgcaaaaaaaaaggagaacacatgcacactaaaaagataaaactgtagagtcctgttgtcaaaatactcaattgtcctttagaccatgtctaactgttcatttatatgattctctaaaacactgatattattgtagtactatagattatattattcgtagagtaaagtttaaatatatgtataaagatagataaactgcacttcaaacaagtgtgacaaaaaaaatatgtggtaattttttataacttagacatgcaatgctcattatctctagagaggggcacgaccgggtcacgctgcactgcaggcatgcaagcttgaattcctgcagccccgccaagctatcaactttgtatagaaaagttggtttgtgtcttctagattaatcctccaaacttttgattaaccaaaaaaattatcaaactaacatgttctccttttttctttagaaattctaacgaatttatctttatactgatttgaatatacttaatttggtcatttggatgccctttacaacctccttaccaaactcactatggcaaatatatactattttccattgtaacataaatgtccataatttgaattaaattcgttgcagtacgaaaccatccaactttgtccaaaaacaaaatccttataactatttactttaatgtaaatatatcctctacttttgtttttacaaccctagctcaaacaaatttattatttgcgataaaaaatcatatcgaacaaactcgatgattttttttttcttacgttattaatgaaactaaaatatagaaaaaaacaagatgaaccaaattttcacctatctaactacttaaatataatatgattaaatttggtaaagtttgaaaagtttctttagaaatgtgaaatattgatcacagtttctattgctaaaatcaccaacaaaacgcatgtcgccattcataattatggtttcacacctacaactaggctaataagtaaataagtagacaactagactcaggtttgaaaaaaccataaaagccatatagcgttttctcattgaaactgcgaacacgatcgtgtgaatgttgcagtttctagttttgatacaaacaaacaaaaacacaatttaatcttagattaaaaagaaaaaagagaacggagcccactagccactccttcaaacgtgtcttaccaactctcttctagaaacaaattaggcttcaccttcctcttccaacctctctctctctctctctctctttttctcaaaccatctctccataaagccctaatttcttcatcacaagaatcagaagaagaaacaagtttgtacaaaaaagcaggcttactgcaaaaaacttatggacctgcatctaattttcggtccaacttgcacaggaaagacgacgaccgcgatagctcttgcccagcagacagggcttccagtcctttcgcttgatcgggtccaatgctgtcctcaactatcaaccggaagcggacgaccaacagtggaagaactgaaaggaacgacgcgtctctaccttgatgatcggcctctggtggagggtatcatcgcagccaagcaagctcatcataggctgatcgaggaggtgtataatcatgaggccaacggcgggcttattcttgagggaggatccacctcgttgctcaactgcatggcgcgaaacagctattggagtgcagattttcgttggcatattattcgccacaagttacccgaccaagagaccttcatgaaagcggccaaggccagagttaagcagatgttgcaccccgctgcaggccattctattattcaagagttggtttatctttggaatgaacctcggctgaggcccattctgaaagagatcgatggatatcgatatgccatgttgtttgctagccagaaccagatcacggcagatatgctattgcagcttgacgcaaatatggaaggtaagttgattaatgggatcgctcaggagtatttcatccatgcgcgccaacaggaacagaaattcccccaagttaacgcagccgctttcgacggattcgaaggtcatccgttcggaatgtattaggtacccagctttcttgtacaaagtggagtccgcaaaaatcaccagtctctctctacaaatctatctctctctatttttctccagaataatgtgtgagtagttcccagataagggaattagggttcttatagggtttcgctcatgtgttgagcatataagaaacccttagtatgtatttgtatttgtaaaatacttctatcaataaaatttctaattcctaaaaccaaaatccagtgacctcaacttta